(19) United States
(12) Patent Application Publication
(10) Pub. No.: US 2009/0011417 A1
(43) Pub. Date: Jan. 8, 2009

(54) Testing Device

(76) Inventors: George Maltezos, Fort Salonga, NY (US); Axel Scherer, Laguna Beach, CA (US); Jingqing Huang, Pasadena, CA (US); Alex Dickinson, Laguna Beach, CA (US); Matthew Johnston, Woodbridge, CA (US)

Correspondence Address: WILSON SONSINI GOODRICH & ROSATI, 650 PAGE MILL ROAD, PALO ALTO, CA, 94304-1050, US

(21) Appl. No.: 12/044,922

(22) Filed: Mar. 7, 2008

Related U.S. Application Data
(60) Provisional application No. 60/905,464, filed on Mar. 7, 2007.
(60) Provisional application No. 60/905,789, filed on Mar. 8, 2007.

(51) Int. Cl. C12Q 1/68; C12M 1/00; C12M 1/24; G06Q 90/00; C12M 1/33; C07H 21/00

(52) U.S. Cl. 435/6; 435/309.1; 435/289.1; 435/304.1; 435/306.1; 536/25.41; 705/500

(57) ABSTRACT

The invention provides, in different aspects, a system, sample preparation device, sample processing cartridge, kit, methods of use, business methods, and computer program product.

### CROSS-REFERENCE

This application claims the benefit of U.S. Provisional Application No. 60/905,464, filed Mar. 7, 2007, U.S. Provisional Application No. 60/905,789, filed Mar. 8, 2007, which are incorporated herein by reference in their entirety.

### STATEMENT AS TO FEDERALLY SPONSORED RESEARCH

This invention was made with the support of the United States government under Contract number ______ by.

### BACKGROUND OF THE INVENTION

The medical diagnostics industry is a critical element of today's healthcare infrastructure. At present, however, diagnostic analyses involving nucleic acid analysis are time consuming and labor intensive. Various reasons contribute to these issues. First, there are usually several steps in a diagnostic analysis between sample collection and obtaining a diagnostic result that require skilled operators, and complex equipment. For example, a biological sample, once extracted from a patient, must be purified to a level compatible with diagnostic assays such as those involving polymerase chain reactions (PCR) to amplify a nucleotide of interest. Once amplified, the presence of a polynucleotide sequence of interest needs to be determined. Sample preparation can be automated, but in practice is routinely carried out by hand. Many diagnostic tests are typically performed with specialized equipment that is both expensive and only operable by trained technicians. The multiple separate steps and reagents used in processing samples to isolate nucleic acids and/or analyze them provides multiple chances for error to occur via operator mistake or reagent contamination/expiration. For example, some detection methods include polynucleotide amplification by polymerase chain reaction (PCR) or a related amplification technique. Such techniques use a cocktail of ingredients, including one or more of an enzyme, a probe, and a labeling agent. Therefore, detection of polynucleotides can require use of a variety of different reagents, many of which require sensitive handling to maintain their integrity, both during use, and over time.

There thus remains a considerable need for methods devices and systems that provide for the isolation of nucleic acids from biological samples and/or the analysis of the resulting nucleic acids in a rapid and simple format.

### SUMMARY OF THE INVENTION

In one aspect the invention provides for a sample preparation device comprising: a housing comprising: a) at least one first input port adapted to engage at least one positive pressure device adapted to deliver at least one fluid reagent into said first input port; b) a processing component; c) at least one first channel in fluid communication between said at least one first input port and said processing component; d) at least one second channel in fluid communication between said processing component and i) a waste port, or ii) a waste chamber; e) at least one third channel in fluid communication between said processing component and at least one collection port; and e) a valve having at least three positions, wherein the first position diverts fluid from the processing component to the waste port or waste chamber, the second position diverts fluid from the processing component to the collection port, and the third position prevents all flow from the processing component. In one embodiment the housing is adapted to receive said processing component. In another embodiment the processing component is integrated into said housing. In another embodiment at least one positive pressure device engaged with said first inlet port. In another embodiment at least one positive pressure device is integrated into said housing. In another embodiment a second input port is adapted to engage at least one second positive pressure device and a fourth channel in fluid communication between said second input port and said collection port without passing through said processing component; and wherein said valve has a third position that diverts fluid from the second input port to the collection port. In another embodiment at least one positive pressure device is a syringe. In another embodiment the housing one of said syringe comprises a dual chamber. In another embodiment at least one positive pressure device comprises a dual chamber. In another embodiment the housing comprises a plurality of first input ports, each engaged with a positive pressure device. In another embodiment the positive pressure devices each comprise at least one different reagent. In another embodiment the at least one collection port further comprises an outlet adapter that fits to a container. In another embodiment the container is a collection vessel or reaction chamber. In another embodiment the outlet adapter includes but is not limited to a luer lock, snap lock, friction fit, grooved screw lock.

In another embodiment the container is a capillary tube, conical tube, well, or PCR tube. In another embodiment the processing component is adapted for nucleic acid purification, protein purification, or chemical compound purification. In another embodiment the waste chamber is further linked to a waste port. In another embodiment the waste chamber or waste port further comprises an aerosol filter. In another embodiment the sample preparation device further comprises a data storage capability.

In one embodiment the data storage component comprises a flash memory card. In another embodiment the sample preparation device comprises a plurality of (e.g., 2, 3, 4, 5 or 6) compartments engaged with input ports, wherein one compartment is adapted to receive a biological sample, another compartment comprises a cell lysis buffer, yet another component comprises wash buffer and yet one more compartment comprises elution buffer; and wherein said second positive pressure device comprises DNA primers and DNA polymerase. As described herein, each compartment can be configured for application of positive pressure (e.g., automated piston, syringe), or for vacuum pressure, where a vacuum is applied to the proximal end of one or more of the plurality of compartments thus drawing the contents of the compartment through the SPD.

In another aspect, the invention provides for a kit comprising a sample preparation device comprising: a housing comprising: a) at least one first input port adapted to engage at least one positive pressure device adapted to deliver at least one fluid reagent into said first input port; b) a processing component; c) at least one first channel in fluid communication between said at least one first input port and said processing component; d) at least one second channel in fluid communication between said processing component and i) a waste port, or ii) a waste chamber; e) at least one third channel in fluid communication between said processing component and at least one collection port; and e) a valve having at least three positions, wherein the first position diverts fluid from the processing component to the waste port or waste chamber, the second position diverts fluid from the processing component to the collection port, and the third position prevents all flow from the processing component; integrated positive pressure devices for fluid delivery; and a sealed pouch.

In another aspect, the invention provides for a kit comprising a sample preparation device comprising: a housing comprising: a) at least one first input port adapted to engage at least one positive pressure device adapted to deliver at least one fluid reagent into said first input port; b) a processing component; c) at least one first channel in fluid communication between said at least one first input port and said processing component; d) at least one second channel in fluid communication between said processing component and i) a waste port, or ii) a waste chamber; e) at least one third channel in fluid communication between said processing component and at least one collection port; and e) a valve having at least three positions, wherein the first position diverts fluid from the processing component to the waste port or waste chamber, the second position diverts fluid from the processing component to the collection port, and the third position prevents all flow from the processing component; syringes comprising reagents; and a sealed pouch

In another aspect, the invention provides for a method of isolating a nucleic acid comprising: a) delivering a sample through a first input port of the module of claim 1 into the processing component; b) lysing said sample and capturing one or more nucleic acids in said processing component; c) washing said captured nucleic acids; and d extracting said nucleic acids from said processing component, and e) collecting the extracted nucleic acids out the collection port. In one embodiment the method further comprises the addition of a reaction mix to the extracted nucleic acids in said container.

In another aspect, the invention provides for a sample preparation device comprising: a) a housing comprising: i) a waste chamber ii) a collection port; b) at least 3 syringes adapted to deliver fluid into a processing component; c) a processing component with a material to capture DNA in fluid communication with said syringes; and d) a valve with at least three positions that can deliver fluid into a waste chamber or a collection port. In one embodiment the sample preparation device further comprises an additional syringe in fluidic communication with the collection port. In another embodiment said syringes are empty, or comprise a reagent selected from the group consisting of lysis buffer, wash buffer, elution buffer, and reaction reagents.

In another aspect, the invention provides for a sample preparation device comprising: a housing comprising: a) at least one first input port adapted to engage at least one reagent reservoir b) a processing component c) at least one first channel in fluid communication between said at least one first input port and said processing component; d) at least one second channel in fluid communication between said processing component and i) a waste port, or ii) a waste chamber; e) at least one third channel in fluid communication between said processing component and at least one collection port; e) a valve having at least three positions, wherein the first position diverts fluid from the processing component to the waste port or waste chamber, the second position diverts fluid from the processing component to the collection port, and the third position prevents all flow from the processing component; and f) at least one pressure port adapted to engage a negative pressure device adapted to deliver at least one fluid reagent into said first input port.

In another aspect the invention provides for a method of distributing the sample preparation device comprising: a housing comprising: a) at least one first input port adapted to engage at least one positive pressure device adapted to deliver at least one fluid reagent into said first input port; b) a processing component; c) at least one first channel in fluid communication between said at least one first input port and said processing component; d) at least one second channel in fluid communication between said processing component and i) a waste port, or ii) a waste chamber; e) at least one third channel in fluid communication between said processing component and at least one collection port; and e) a valve having at least three positions, wherein the first position diverts fluid from the processing component to the waste port or waste chamber, the second position diverts fluid from the processing component to the collection port, and the third position prevents all flow from the processing component; to a distributor; wherein said distributor provides one or more positive pressure devices loaded with one or more reagents; wherein said distributor sells or licenses said sample preparation device and said one or more positive pressure devices. In one embodiment the sample preparation device comprises a data storage capability. In another embodiment the distributor provides one or more positive pressure devices loaded with one or more reagents and one or more computer programs to the data storage capability; wherein said distributor sells or licenses said sample preparation device and said one or more positive pressure devices.

In another aspect the invention provides for a method of rapid pathogen detection comprising: processing a biological sample with the sample preparation device of claim 1; delivering at least one nucleic acid sequence and a reaction mix to a collection vessel; and analyzing said at least one nucleic acid sequence in a liquid metal thermal cycler comprising an optical assembly.

In various embodiments, compositions and methods are provided for improved and simplified distribution of disposable, self-contained or alternatively semi-self-contained cartridges configured to isolate a target compound. Cartridges comprise all the necessary reagents, buffers, enzymes for conducting an assay (e.g., PCR) and can be further configured to be operably linked to a second device or machine as further described herein. In addition, such cartridges can be stored and transported with or alternatively without compartments comprising the necessary buffers, reagents and solvents necessary to obtain a target molecule from a sample. For example, a cartridge can comprise a plurality of compartments containing the necessary reagents a single unit or can be configured to receive such compartments. Furthermore, cartridges can be distributed, sold, transported or stored with a data storage component which is capable of uploading or downloading data or computer executable logic.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the components of an embodiment of a sample preparation device

FIG. 2 illustrates the operation of a sample preparation device: A, a sample preparation device ready for use, comprising a syringe loaded with a sample comprising nucleic acids; B, the syringe loaded with a sample comprising nucleic acids and a dual chamber syringe comprising solvent and lyophilized lysis reagents are depressed; C, wash buffer is delivered to the processing component in two stages by the depression of two different syringes; D, a valve on the housing is rotated so as to block the channel leading to the waste chamber and open access to the channel leading to a collection vessel; E, Elution buffer is then delivered to the processing component by the depression of a fifth syringe, which elutes nucleic acids (such as DNA or RNA) and is delivered to a collection vessel; F, a valve on the housing is rotated so as to block the channel leading to the waste chamber and the channel leading to the collection vessel; G a sixth dual chamber syringe comprising lyophilized reagents and solvent is depressed, delivering reconstituted reagents to the collection vessel.

FIG. 3 A, illustrates the external features of another embodiment of a sample preparation device; B illustrates the internal schematics of an embodiment of a sample preparation device.

FIG. 4 illustrates the perspective of another embodiment of a sample preparation device.

FIG. 5 illustrates samples processed using a SPD (Sample 1) and a conventional system (Sample 2).

FIG. 6 illustrates samples processed using a SPD (Sample 1) and a conventional system (Sample 2).

FIG. 7 illustrates a sample collection device operably linked to a PCR machine.

FIG. 8 illustrates a sample collection device operably linked to a PCR machine.

### DETAILED DESCRIPTION OF THE INVENTION

The invention provides, in different aspects, a system, sample preparation device, sample processing cartridge, kit, methods of use, business methods, and computer program product, are now further described. In general, the devices and methods of the invention provide for rapid and simplified processing of a sample from an organism. The sample preparation device (“SPD”) provides a single housing which comprises all the necessary components to process a sample to obtain a desired target compound, such as a nucleic acid molecule. In addition, the SPD is configured to receive or have integrated within a collection vessel. The SPD can comprise reagents necessary for subsequent processing of a target molecule which is eluted into the collection vessel. Alternatively, the collection vessel itself can comprise additional reagents (e.g., lyophilized or gelified), which are reconstituted upon contact with a solution comprising a target compound. For example, the collection vessel can comprise reagents necessary for a reaction (e.g., PCR), thus can be subjected to PCR.

Furthermore, the SPD can be configured to comprise a data storage component (“DSC”) which can store data related to sample processing or reagents used therefore, as well as contain computer executable that can function to provide instructions for conducting a particular assay or operation of another device (e.g., PCR machine) per a protocol contained on the DSC. The DSC can be configured to upload or download data and/or computer executable logic through conventional wireless or wired technology.

Analysis of biological samples often includes determining whether one or more polynucleotides (e.g., a DNA, RNA, mRNA, or rRNA) can be present in the sample. For example, one may analyze a sample to determine whether a polynucleotide indicative of the presence of a particular pathogen (such as a bacterium or a virus) can be present. The polynucleotide may be a sample of genomic DNA, or may be a sample of mitochondrial DNA.

Typically, biological samples which can be processed using a SPD of the invention can be complex mixtures. For example, a sample may be provided as a blood sample, a tissue sample (e.g., a swab of, for example, nasal, buccal, anal, or vaginal tissue), a biopsy aspirate, a lysate, as fungi, or as bacteria. Polynucleotides to be determined may be contained within particles (e.g., cells (e.g., white blood cells and/or red blood cells), tissue fragments, bacteria (e.g., gram positive bacteria and/or gram negative bacteria), fungi, spores). One or more liquids (e.g., water, a buffer, blood, blood plasma, saliva, urine, spinal fluid, or organic solvent) can typically be part of the sample and/or can be added to the sample during a processing step.

Methods for analyzing biological samples include providing a biological sample (e.g., a swab or a fluid), releasing polynucleotides from particles (e.g., cell lysis) of the sample, amplifying one or more of the released polynucleotides (e.g., by polymerase chain reaction (PCR)), and determining the presence (or absence) of the amplified polynucleotide(s) (e.g., by fluorescence detection).

### Sample Preparation Device

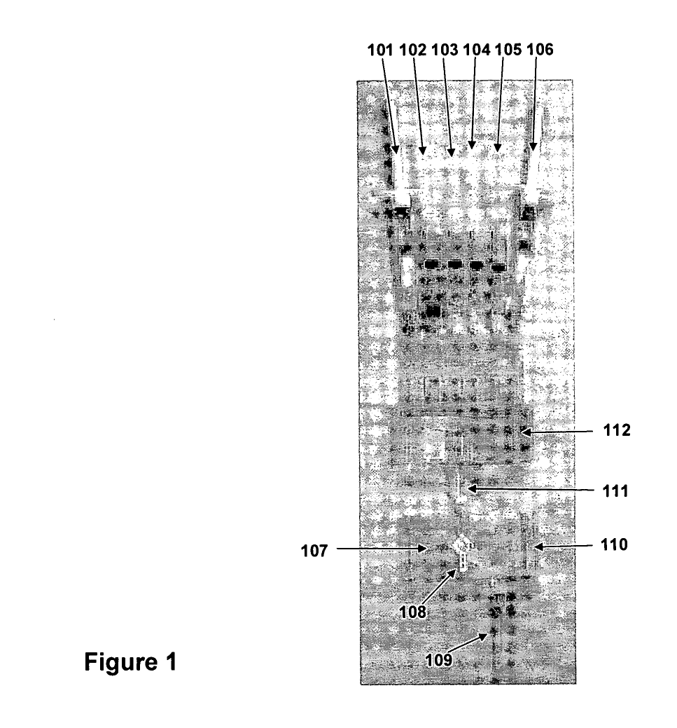

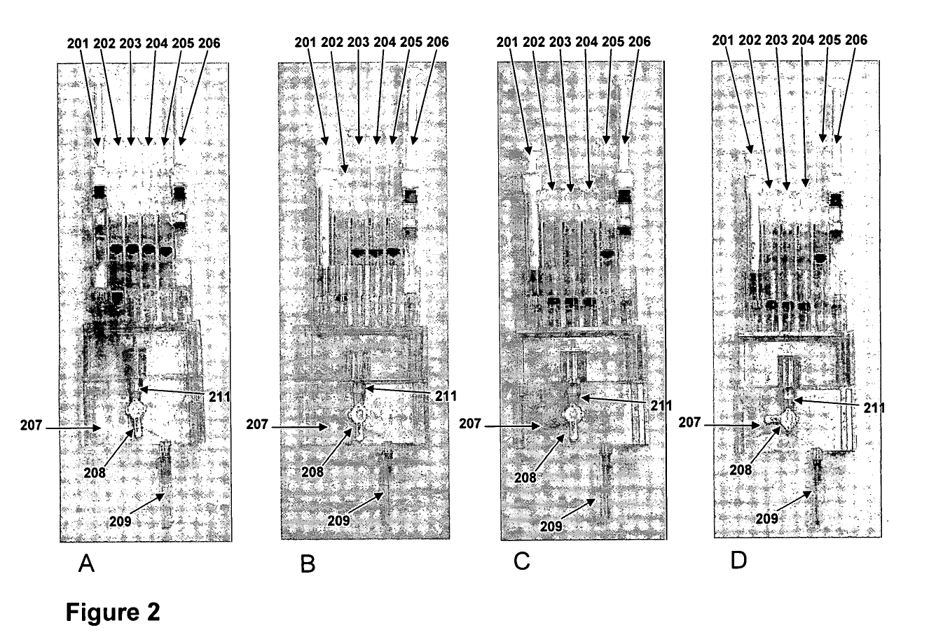

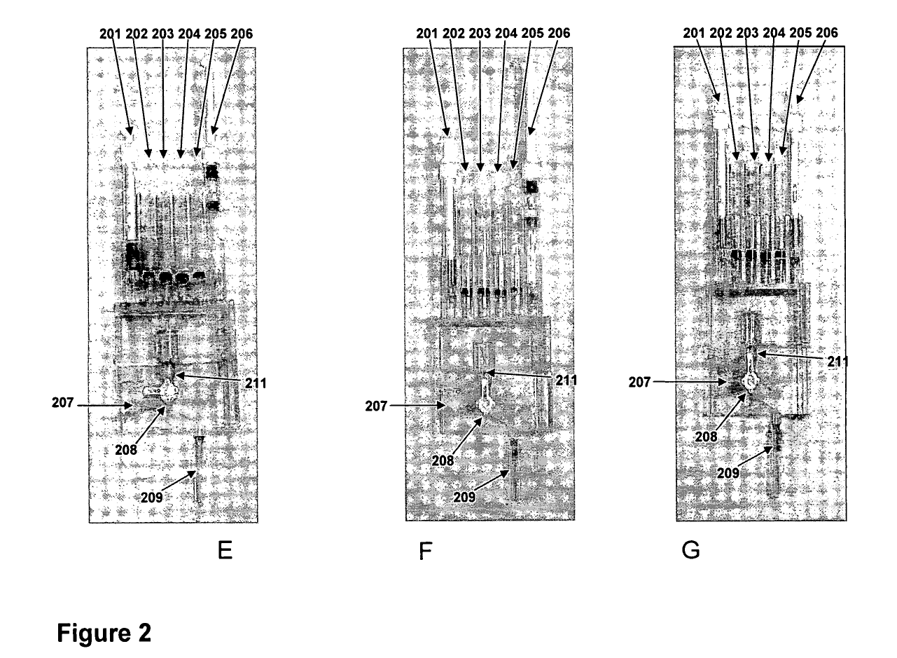

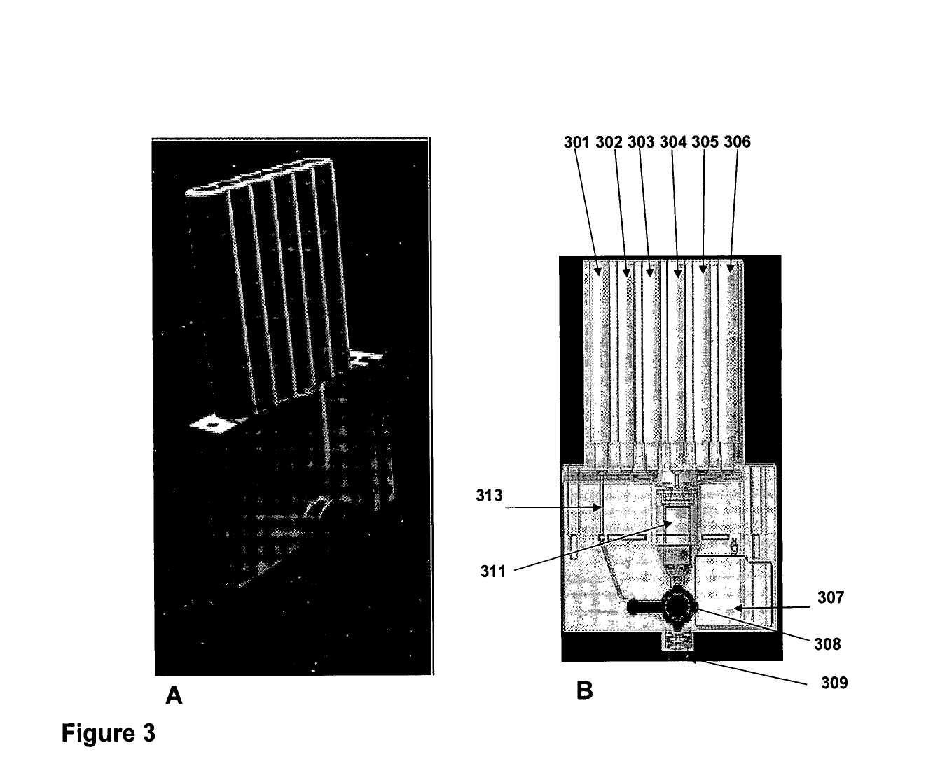

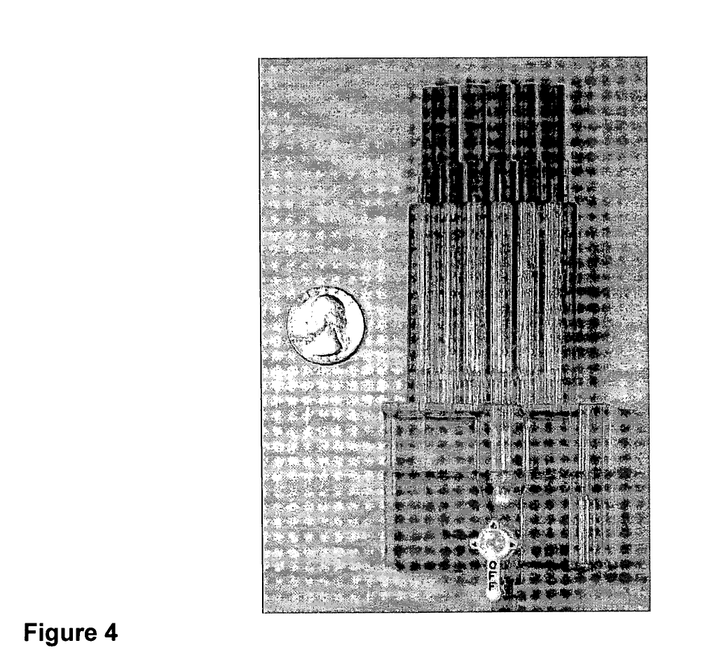

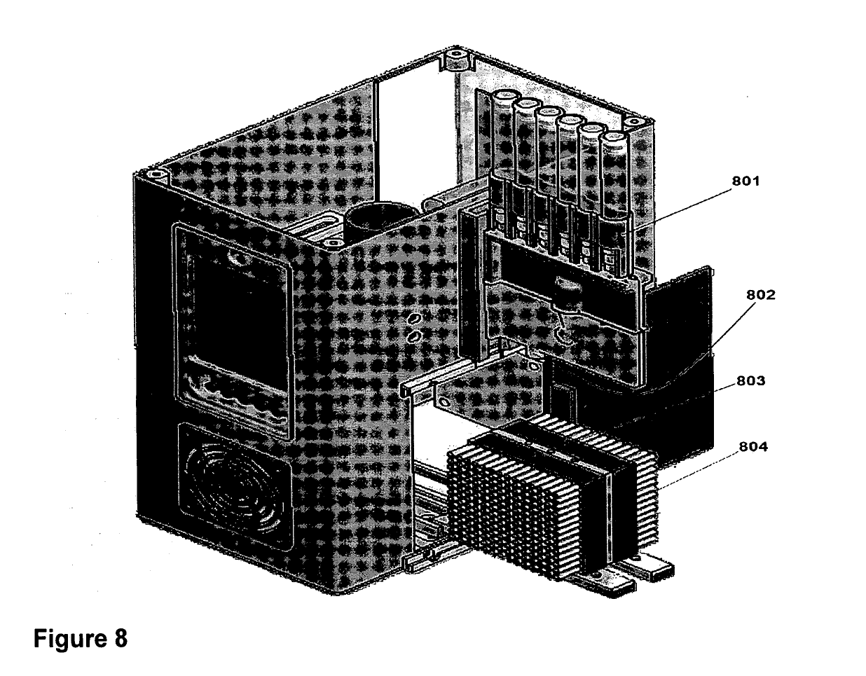

In one aspect of the invention a sample preparation device (SPD) is provided for processing a sample to isolate a target compound. In various embodiments, the SPD comprises one or more integrated compartments 101-106 or is configured to receive on or more compartments 101-106. As further described herein, the compartments can be configured for positive pressure (e.g., piston, syringe), negative pressure (e.g., vacuum) or a compartment that is pressurized and sealed, where release of the contents is effected through puncture of the seal. The SPD (e.g., FIGS. 1 and 2) can be configured for fitting or attachment to other devices or compartments. For example, as depicted in FIG. 8 the SPD 801 is fitted through a slide and groove means to a PCR machine. The SPD can comprise a collection vessel 109, 703 or a collection vessel can be attached to the outlet port 309, 802. As is described herein, a SPD provides a single, self-contained device which can be stored and shipped without the need for cold storage, for storing multiple wash buffers, reagents, solvents and other components necessary for isolation of a target compound. Methods for providing stabilized storage of reagents and biologicals which can be adapted for use in the various aspects of the invention are disclosed in U.S. Patent Application Publication Nos. 20080050737; 20070172875; 20070207956; 20070110809; and 20060275886.

Furthermore, in yet further embodiments, a SPD comprises a DSC as described herein, which can store data, comprise computer executable logic (software) to operate additional devices operationally linked to the SPD, and/or perform analysis on data or components related to a sample processed by the SPD.

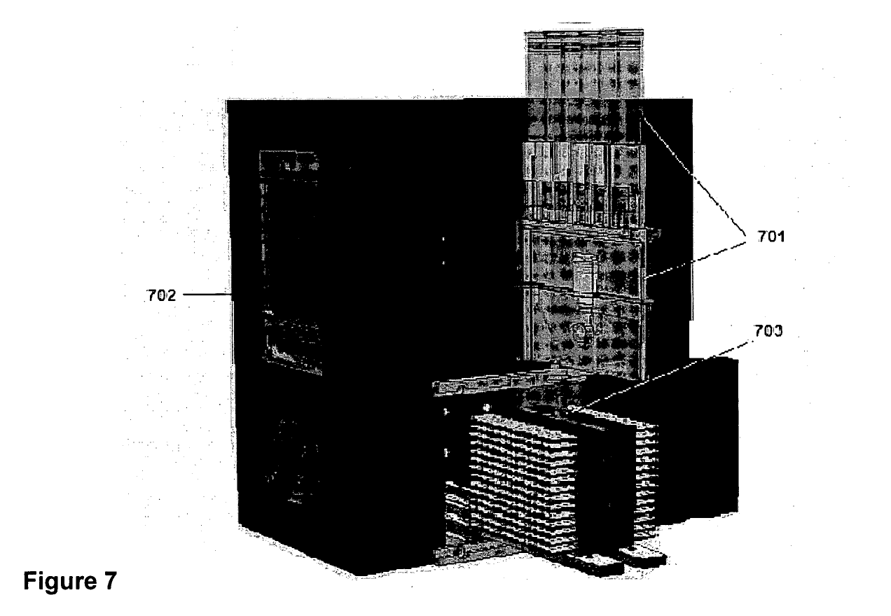

The terms “operationally linked”, “operably linked”, “operatively linked” or variations thereof as used herein, mean in the particular context used, that one component is linked to another component. For example, if a collection vessel is integrated into or fitted to a SPD, then it is “operably linked” in the sense that the contents of a SPD can be flowed into the collection vessel. In another example, a SPD can be operably linked to a PCR machine (FIGS. 7 and 8).

As used herein the terms cartridge, sample collection device cartridge and SPD may be used interchangeably.

Depending on the particular target molecule sought to be isolated from a given sample, the processing component 111, 311 can be a different component (e.g., designed for isolation of RNA, DNA, protein, carbohydrates, lipids). Furthermore, a SPD can have such a processing component integrated at the time of manufacture or production, or configured to receive a processing component subsequently (e.g., by an end-user, distributor).

In one embodiment the SPD is designed to isolate one or more nucleic acids such as RNA or DNA from a sample. In another embodiment the sample preparation device is designed to isolate one or more proteins. In another embodiment the sample preparation device is designed to isolate one or more lipids. In yet another embodiment the sample preparation device is designed to isolate one or more polysaccharides. Depending on the compound to be isolated, the SPD is configured to comprise various reagents, buffers and solvents conventional to isolation of the particular compound, from a particular sample. The SPD provides a plurality of compartments, each of which can be configured to contain a necessary reagent, buffer or solvent. For example, if the desired compound (also “target compound”) is a nucleic acid and the sample is a blood sample, the SPD is configured with the necessary lysis buffers (e.g., to lyse cells in the sample), wash buffers and solvents. Furthermore, as described herein, the SPD is configured to contain or receive a processing means which provides for isolation of the desired compound (e.g., a DNA purification column to isolate target nucleic acids). Thus, in this example, the SPD is configured to comprise wash buffers and a collection buffer that provides the target compound in a collection solution (e.g., buffer containing nucleic acids). Furthermore, the SPD can be configured to provide additional reagents for downstream processing of the target compound. For example, the SPD is configured to provide reagents necessary for subsequent reactions involving the target compound (e.g., reagents, primers, buffers for PCR). As will be evident from the descriptions herein, the SPD provides means for compartmentalizing a plurality of different ingredients necessary to isolate a given target compound, as well as further downstream analysis and processing of target compounds.

In one embodiment the SPD comprises one or more delivery units or reagent reservoirs; a housing, comprising a processing component, conduits (including but not limited to a capillary channel, a channel or a channel), a waste chamber or waste port, and a collection port; and, optionally, a collection vessel.

In some embodiments the delivery units comprise reagent delivery units and/or sample delivery units. In some embodiment the delivery units are positive pressure devices, including but not limited to syringes, pipettes, or pump driven devices. In some embodiments the delivery units are negative pressure delivery units, such as receptacles, which are evacuated by vacuum pressure into the housing. In one embodiment the SPD comprises one or more (such as 2, 3, 4, 5, 6 7, 8, 9, 10, 12, 18, 24, 30, 36, 42 or 48) sample delivery units. In one embodiment the SPD comprises 1 or more (such as 2, 3, 4, 5, 6 7, 8, 9, 10, 12, 18, 24, 30, 36, 42, 48, 54, 60, 66, 72, 78, 84, 90, 96 or 102) reagent delivery units. In another embodiment t the SPD comprises 5 times as many reagent delivery units as there are sample delivery units. In one embodiment the one or more reagent delivery units are integrated with the housing. In another embodiment the one or more reagent delivery units are removable from the housing. In one embodiment the housing comprises at least on input port adapted to connect to at least one reagent delivery unit and/or sample delivery unit. In another embodiment, at least one delivery unit is connected to a housing by a connector such as a threaded connector (for example a Luer lock).

The term “fluidic” as used herein includes microfluidic and mesofluidic volumes.

In some embodiments the reagent delivery units are plunger driven, such as syringes. In some embodiments one or more of the delivery units (such as a syringe or a pipette) delivers at least one reagent, including but not limited to, lysis buffers (wherein the lysis buffer (such as TR-HCL) may comprise one or more lysis reagents (such as enzymes or surfactants (e.g., Triton X or NP40)), one or more salt solutions, or EDTA, a DNAse inhibitor, an RNAse inhibitor or a protease inhibitor), wash buffers (such as high or low salt wash buffers), and elution buffers (such as deionized water or EDTA elution buffers). In one embodiment the reagents are stable at room temperature. In one embodiment the reagents are stable for 1-6 months. In another embodiment the reagents are stable for at least 6 months in another embodiment the reagents are stable for at least 12 months. In one embodiment the reagents are lyophilized and are reconstituted with a diluent or solvent prior to use. In some embodiments one or more of the delivery units (such as a syringe or a pipette) delivers a reaction mix that comprises all of the reagents necessary to perform a reaction such as polymerase chain reaction (PCR), quantitative polymerase chain reaction (qPCR), nucleic acid sequencing, ligase chain polymerase chain reaction (LCR-PCR), reverse transcription PCR reaction (RT-PCR), single base extension reaction (SBE), multiplex single base extension reaction (MSBE), reverse transcription, and nucleic acid ligation. In another embodiment one or more of the delivery units (such as a syringe or a pipette) comprises a reaction mix, including but not limited to one or more of the following: a PCR master mix (comprising one or more components such as DNA polymerase, dNTPs, buffer, Mg+, primers, labeled primers, or fluorophores), a reverse transcription master mix (comprising one or more components such as DNA polymerase, reverse polymerase, dNTPs, buffers, Mg+, primers, labeled primers, or fluorophores), a real-time PCR master mix (comprising one or more components such as DNA polymerase, dNTPs, buffer, Mg+, primers, labeled primers, or fluorophores), sequencing reaction mix (comprising one or more components such as DNA polymerase, labeled dNTPs, buffers, Mg+, a primer, and fluorophores), a restriction mix (comprising one or more components such as a restriction enzyme, buffer and a salt solution) or a ligation mix (comprising one or more components such as a ligation enzyme, buffer).

Gelification is a process where components are stabilised at room temperature by the addition of different stabilising agents. This process does not alter protein structures and interaction between reagents are avoided until reaction is activated by the user. This technology can be applied to a variety of enzymatic reactions and proteins, such as antibodies, used in molecular biology research, development and diagnosis. Gelification represents a step forward in comparison to other methods for the stabilisation of reaction mixes, such as lyophilisation, heat dissecation and agarose beads. Gelification is simple, efficient and economical.

In one embodiment each reagent is contained within a single delivery unit, such as a dual-chamber syringe (such as the Lyoject syringe). In one embodiment the SPD comprises at least 2 dual-chamber syringes (such as 3, 4, 5 6, 7, 8, 10 or 12). In further embodiments, the SPD comprises one or more three-chamber component (e.g., syringe). Thus, in various embodiments, the multichamber component (e.g., dual- or three-chamber syringe) comprises a reagent that is lyophilized in one chamber, while the other chamber (e.g., in a dual chamber syringe) contains a solvent or reconstitution fluid that is mixed with the active substance immediately before application to the sample preparation device. Multi-chamber components of the SPD can be adapted to contain additional buffers, reagents, solvents or additives as desired (e.g., a third chamber can comprise an additional buffer, such as a lysis buffer, or another reagent, etc.).

In one embodiment the SPD comprises one or more sample delivery units. In some embodiments the one or more sample delivery units are plunger driven, such as syringes. In some embodiments the sample delivery unit can be removed from the housing for sample loading. In one embodiment the housing comprises at least one input port adapted to connect to at least one sample delivery unit. In another embodiment the sample delivery unit is integrated with the housing and comprises an input for delivering the sample to the sample delivery unit.

In some embodiments, the SPD comprises at least one probe that can bind a polynucleotide sequence, wherein the SPD can be configured to contact a polynucleotide sample or a PCR amplicon thereof with the probe. In some embodiments the probe is bound to a substrate (such as wall of the SPD or a microbead). In another embodiment the probe comprises a label, such as a fluorophore. In one embodiment the probe is a fluorescent oligonucleotide probe. In another embodiment the fluorescent oligonucleotide probe comprises a polynucleotide sequence coupled to a fluorescent reporter dye and a fluorescence quencher dye. In another embodiment the probe comprises a chromogenic label. In some embodiments the PCR reagents can further comprise a positive control plasmid and/or a plasmid fluorescent oligonucleotide probe selective for at least a portion of the plasmid. In one embodiment the system comprising the SPD can be configured to allow independent optical detection of the fluorescent oligonucleotide probe and the plasmid fluorescent oligonucleotide probe.

In some embodiments, the probe binds to a polynucleotide sequence that is characteristic of an organism. For example a probe can bind to deoxyribonucleic acid or ribonucleic acid polynucleotide sequence that is specific for an organism. In this manner if a probe binds a sequence in a sample than this can indicate the presence of a specific organism.

In some embodiments a probe binds to a deoxyribonucleic acid or ribonucleic acid polynucleotide sequence from a biological sample from an organisms such as a mammal (including, but not limited to humans, dogs, cats, horses, apes, elephants, giraffes, monkeys, baboons, deer, cows, pigs, goats, sheep, rats, mice, rabbits, or donkeys), birds (including, but not limited to, chickens, turkeys, geese, partridges or game hens), reptiles (including, but not limited to, snakes, lizards, or toads), amphibians (including, but not limited to, frogs), fish (including, but not limited to, salmon, cod, herring, sardines, Patagonian tooth fish, flounder, sole, or tuna), crustaceans (shrimp, lobster, crabs, prawns), domesticated animals, farmed animals, wild animals, extinct organisms, bacteria, fungi, viruses, or plants. In some embodiments a probe can bind a polynucleotide sequence specific for a sub-cellular organelle of an organism (such as mitochondria or chloroplasts). In some embodiments, the probe can bind a polynucleotide sequence specific for a microorganism. For example, microorganisms used in food production (including, but not limited to, yeasts employed in fermented products, molds or bacteria employed in cheeses) or pathogens (including, but not limited to, E. coli, Staphylococcus, Streptococcus, Anthrax, HIV, Herpes simplex, Cytomegalovirus, Influenza, Cholera, or Tuberculosis). In some embodiments, the probe can bind a polynucleotide sequence specific for organisms selected from the group consisting of gram positive bacteria, gram negative bacteria, yeast, fungi, protozoa, and viruses. In various embodiments, the probe can bind a polynucleotide sequence specific for Group B Streptococcus. In some embodiments, the SPD can be configured to allow optical detection of the fluorescent oligonucleotide probe.

In some embodiments a probe binds to a deoxyribonucleic acid sequence from a specific chromosome. In one embodiment a probe binds to a specific gene sequences. In another embodiment a probe binds to a specific allele sequences of specific genes. In another embodiment a probe binds to a ribonucleic acid polynucleotide sequence from a biological sample from an organism.

In one embodiment a sample is loaded into a dual or triple chamber delivery unit that comprises one or more reagents such as lyophilized lysis reagents and solvent in separate chambers, or lysis buffer in a top chamber and a sample loading chamber in the bottom. In this embodiment the sample can be loaded into the bottom chamber of a delivery unit, such as a syringe. Then the syringe can be coupled to the housing of the nucleic acid sample preparation device. Next the sample can be delivered to a processing component, followed by the lysis buffer.

In one embodiment the housing comprises at least one channel. In some embodiments, the housing comprises a channel connected to at least one vent. In some embodiments at least one channel fluidly connects one or more delivery units to a processing component. In another embodiment at least one channel fluidly connects two or more syringes to a processing component. In another embodiment each delivery unit is fluidly connected to a channel which in turn is fluidly connected to a processing component.

In another embodiment at least one channel fluidly connects one or more delivery units to a collection port. In another embodiment at least one channel fluidly connects one or more syringes to a collection port. In one embodiment at least one channel fluidly connects the processing component to a waste chamber. In an alternative embodiment at least one channel fluidly connects the processing component to a waste port. In one embodiment the housing comprises a valve which can be used to divert fluid from the processing component to a channel fluidly connected to a waste port, a waste chamber or a collection vessel. In another embodiment the valve comprises an off position that blocks all fluid flow from the processing component. In another embodiment the valve comprises an off position that blocks or prevents fluid backflow into the processing component when a reagent mix is delivered to a collection vessel from a delivery unit. For example see FIGS. 2F & G. In some embodiments, the collection vessel itself comprises lyophilized or gelified reagents necessary for a particular reaction (e.g., PCR). Thus, the collection vessel itself can function as a reaction compartment. In other embodiments, the necessary reagents for subsequent processing of the target compound are provided in one of the compartments in the SPD (106, 206, 301).

In one embodiment the housing comprises an access opening into which the processing component can be inserted. In another embodiment the access is built into the housing. In another embodiment the housing can be separated into two or more pieces exposing an internal opening that can accept the processing component. In another embodiment the nucleic acid sample preparation device comprises a processing component integrated into the housing of said SPD. In one embodiment the SPD is a single use device. In one embodiment the processing component comprises one or more nucleic acid capture materials (including, but not limited to, glass fiber, nitrocellulose, or hydroxyapatite). In another embodiment the processing component comprises one or more nucleic acid binding materials, including but not limited to, ferrous or polystyrene beads coupled to a nucleic acid binding moiety, or a one or more nucleic acid binding moieties bound to a substrate such as one or more walls of the processing component. In one embodiment the processing component comprises a filter, which may comprise one or more materials intended to capture nucleic acids. In one embodiment the processing component is a QIAamp Mini Spin column. In one embodiment the processing component is used for DNA and/or RNA capture. In another embodiment the processing component captures nucleic acids present in a sample.

In one embodiment the housing of the sample preparation device is adapted to connect to a collection vessel via a collection port. In another embodiment, the collection vessel is connected to the housing by a connector such as a threaded connector (for example a Luer lock). In another the collection vessel is fluidly connected to the housing, such as to delivery tube connected to a collection port. In one embodiment the collection vessel comprises at least one reagent such as a nucleic acid buffer, EDTA, sterile water, deionized water, DNA polymerase, reverse polymerase, primers, labeled primers, dNTPs, PCR buffer, Mg+, and fluorophores. In one embodiment the collection vessel is a capillary tube, a conical tube, a reaction tube, a well in multi-well plate, or a fluidic cartridge.

In one embodiment the housing of the nucleic acid sample preparation device is fluidly coupled to an analysis apparatus so that purified nucleic acids or purified nucleic acids and reagents are delivered to said analysis apparatus. In one embodiment purified nucleic acids or purified nucleic acids and reagents are delivered to a collection vessel in an analysis apparatus (including but not limited to a reaction tube, a well on a multi-well plate, a capillary tube, or an SPC). In another purified nucleic acids or purified nucleic acids and reagents are delivered to a channel in an analysis apparatus.

In one embodiment the analysis apparatus comprises a thermal cycler. In one embodiment the thermal cycler is a PCR thermal cycler, such as a liquid metal thermal cycler. In another embodiment the analysis apparatus comprises at least one light source, such as an LED or a coherent light source (e.g. a laser). In another embodiment the analysis apparatus is capable of amplifying at least one nucleic acid sequence and detecting a resulting amplicon. In one embodiment the analysis apparatus detects a amplicon by detecting a florescent dye (including but not limited to Syber green, Syber gold, Thiazole Orange or ethidium bromide) or a fluorophore (including but not limited to, ROX, JOE, FAM, VIC, NED, HEX, Texas Red, TAMRA, Cy-3, or Cy-5). In another embodiment the analysis apparatus is capable of performing a restriction enzyme digestion on at least one nucleic acid sequence. In another embodiment the analysis apparatus is capable of performing a ligation reaction on at least one nucleic acid sequence. In another embodiment t the analysis apparatus is capable of delivering one or more reagents to the purified nucleic acids delivered from the nucleic acid sample preparation device.

In one aspect the nucleic acid sample preparation device is used to prepare a sample for analysis (such as a biological sample). In one embodiment a biological sample may comprise blood, urine, tears, semen, feces, saliva, sputum, a buccal sample, a lung lavage sample, a vaginal sample, amniotic fluid, a hair bulb, or a tissue sample. In another embodiment the sample is selected from the group consisting of a tissue culture, a plasmid sample, a bacteria culture, a viral culture. In another embodiment the sample may be a water sample, an air sample, a food sample, a drug sample, or any other sample to tested for contamination with a microorganism (such as bacteria or viruses). In another embodiment the sample may comprise one or more eukaryotic, prokaryote or viral nucleic acids.

In one embodiment the nucleic acid sample preparation device is used to prepare a purified nucleic acid sequence for testing or analysis. In some embodiments purified refers to the removal of a substantial amount of non-nucleic acid sample components, such as proteins, lipids, polysaccharides and/or salts. In some embodiments purified refers to the removal of a substantial amount of one or more polymerase chain reaction inhibitor selected from the group consisting of hemoglobin, peptides, fecal compounds, humic acids, mucosal compounds, DNA binding proteins, or a saccharide. In one embodiment the nucleic acid sample preparation device is used to prepare a master mix of purified nucleic acids and reagents for analysis. In some embodiments analysis of the purified nucleic acids includes but is not limited to, PCR amplification, Real-Time PCR, Reverse Transcription, DNA sequencing, nucleic acid enzyme digestion, nucleic acid ligation, Transcription, Translation, DNA methylation studies, SNP detection, STR analysis, Microsatellite analysis, RFLP analysis, and DNA fingerprint analysis.

In one example a nucleic acid sample preparation device comprising: 6 syringes; and a housing comprising a processing component and a waste chamber, is used in a method to prepare a purified nucleic acid sequence for PCR (FIGS. 2 & 3). First, a sample comprising nucleic acids is loaded into a syringe (102, 202, 305). Next, the sample syringe and a dual chamber syringe comprising solvent and lyophilized lysis reagents are depressed (101, 102, 201, 202, 305, 306). Next the solutions are allowed to flow-through the processing component (111, 211, 311) into the waste chamber (107, 207, 307) over a period of time (about 10 minutes), wherein one or more nucleic acids present in the sample are captured in the processing component. Next, wash buffer is delivered to the processing component in two stages by the depression of two different syringes (103, 104, 203, 204, 303, 304). As the wash buffer exits the processing component it is delivered to the waste chamber. Next, a valve on the housing (101, 208, 308) is rotated so as to block the channel leading to the waste chamber and open access to the channel leading to a collection vessel (109, 209). Elution buffer is then delivered to the processing component by the depression of a fifth syringe (105, 205, 302). This fluid flows through the processing component, elutes nucleic acids (such as DNA or RNA) and is delivered to the collection vessel (109, 209). Next, a valve on the housing (101, 208, 308) is rotated so as to block both the channel leading to the waste chamber and the channel leading to the collection vessel (off position). Finally, a sixth dual chamber syringe (106, 206, 301) comprising lyophilized PCR reagents and solvent is depressed, delivering reconstituted PCR reagents via channel (313) to the collection vessel (109, 209). The collection vessel can then be removed from the housing and used directly in a method of PCR analysis.

In some embodiments, the SPD comprises one or more lyophilized or stabilized reagents in a reagent reservoir. In some embodiments the reagents comprises all of the reagents necessary for lysing a sample, washing bound nucleic acids and eluting the nucleic acids. In some embodiments, the SPD comprises a lyophilized or stabilized reaction mix in a reagent reservoir. In some embodiments, the SPD comprises a reaction mix to perform a reaction such as polymerase chain reaction (PCR), quantitative polymerase chain reaction (qPCR), nucleic acid sequencing, ligase chain polymerase chain reaction (LCR-PCR), reverse transcription PCR reaction (RT-PCR), single base extension reaction (SBE), multiplex single base extension reaction (MSBE), reverse transcription, or nucleic acid ligation. In one embodiment a reaction mix comprises any number (e.g., 0, 1, 2, or all) of the reagents for performing PCR can be incorporated on the SPD in a lyophilized format. In some embodiments the SPD reaction mix comprises at least one reagent for performing PCR or reverse transcription, including but not limited to DNA polymerase, reverse polymerase, dNTPs, buffer, Mg+, primers, labeled primers, fluorophores, or intercalating dyes. At the time of use, the lyophilized PCR reagents can be reconstituted using, for example, deionized water, which may be stored on the SPD in a blister format (e.g., in a self-pierceable reservoir). In another embodiment the lyophilized PCR reagents can be reconstituted by delivery of a fluid (such as sterile or deionized water, or a buffer) to the SPD via a sample or reagent input port. In some embodiments, the reconstituted PCR reagents can be aliquoted into, two or more aliquots. In some embodiments, the housing of the SPD is connected to a vacuum that provides negative pressure which induces fluid flow from a reagent reservoir into a processing component or into a collection vessel.

In some embodiments, the SPD comprises at least one of a manually actuated pump, a electrically actuated pump, a electrically actuated valve, a thermally actuated pump, a thermally actuated valve, an input port valve, a waste port valve, a collection port valve, at least one filter (such as an aerosol filter), a diaphragm valve, or a reservoir. In some embodiments the diaphragm valve is a Microscale On-chip Valve (MOV) that is actuated by pneumatics (U.S. Pat. No. 6,551,839; U.S. patent application Ser. No. 11/229,065; U.S. Pat. No. 6,190,616; U.S. Pat. No. 6,423,536; U.S. application Ser. No. 09/770,412; U.S. Pat. No. 6,870,185; U.S. application Ser. No. 10/125,045; U.S. application Ser. No. 10/540,658; U.S. patent application Ser. No. 10/750,533; U.S. patent application Ser. No. 11/138,018; all of which are herein incorporated by reference in their entirety). In some embodiments a three MOV valve pump is used to pump fluids through conduits, such as channels, in an SPD. In some embodiments the SPD comprises more than one conduit. The conduits can be independent of each other, or can be partially dependent, for example, the conduits can share one or more reagents such as a lysis reagent.

In some embodiments the SPD comprises a data storage capacity (DSC). In some embodiments the DSC comprises a memory device, which may be integrated into the SPD, or removable. In one embodiment a memory device is a solid state nonvolatile memory such as MRAM, EPROM, EEPROM, NVRAM, FeRAM, STT-MRAM, SONOS, and Flash. In another embodiment the memory device is a hard drive. In another embodiment the memory device is a recordable media, such as optical or magnetic media. In one embodiment the solid state nonvolatile memory used is flash memory. Flash memory is integrated circuit memory that does not need continuous power to retain stored data. It has a limited life span of, for example, 100,000 write cycles. Typical flash memory is erased in blocks of data rather than single bytes of data, thus reducing the erase and write cycle times necessary to store data in such memories. Flash has relatively low cost and can be configured to have a fairly large size. The amount of secondary nonvolatile memory required can vary based on the needs of the host device. For example, flash memory cards in a wide variety of formats are available in sizes ranging from 16 kb to 32 gb.

In one embodiment the SPD comprises a data storage component (DSC), including but not limited to a removable flash memory card, including but not limited to a Secure Digital (SD) card, a Compact Flash (CF) card, a Multi Media Card (MMC), a Smart Media Card (SMC), a Memory Stick, a Memory Stick Pro, a Memory Stick Pro Duo or an xd card. In another embodiment the DSC comprises software, including but not limited to testing programs (e.g., programs to analyze melting curve data or RT-PCR data analysis), calibration programs, verification programs, software updates to the system, or other programs. The DSC is configured to store data and computer executable logic which can be linked through convention means (e.g., hard wire or wireless) to upload/download data and/or program files from a device. In some embodiments of the invention, the DSC will be uploaded with a particular program for operating a device to which the SPD is operably linked (e.g., PCR machine; FIGS. 7 and 8). In some embodiments, the DSC can be configured to comprise specific protocols particular to the assay being conducted or diagnostic test to be run. For example, the DSC can comprise a particular protocol to be run based on the particular pathogen being detected. For example, the DSC can have the protocol parameters for operating a PCR machine 702 that is operably linked to the SPD 701.

In one embodiment the SPD comprises a data storage component (DSC) which contains therein computer executable logic which functions to link the SPD directly to patient specific data. In another embodiment, data is obtained by analysis with the SPD and delivered to a health or research professional. In some embodiments the delivery is automatic. In further embodiments the computer program or software encrypts the data to insure its security.

In various embodiments, SCDs of the invention comprise computer executable logic that functions to achieve processes which include but are not limited to operate the SPD automatically, run analysis on data, run tests on compounds contained therein (e.g., primers, enzymes, chemicals), operate operational protocols (e.g., PCR runs, temperature cycles, etc.). The relevant art in the software, programming or writing computer executable logic is well developed and conventional.

In some embodiments, the SPD can further include a computer-readable label. For example, the label can include an optically readable code, such as a bar code, Dotcode (such as Dotcode-128) a radio frequency tag (RFID tag), one or more computer-readable characters or a smartcard chip (such as a contacted or contact less). Such label(s) can be utilized to track processing of samples, identify samples, identify a particular lot number for SPDs, or identify patients, and any other information that can be stored conventionally on such labels. In some embodiments the SPD comprises a smartcard chip that is cryptographically secure and serves to identify a genuine SPD. In some embodiments the SPD is designed for a single use and the smartcard deauthorizes the fluidic device after one use. In some embodiments the SPD comprises a unique registration number.

In some embodiments the SPD is adapted to be received by a device such as a thermal cycler. In one embodiment the SPD can deliver nucleic acids and optionally reagents to a collection vessel engaged with a thermal cycler. In another embodiment the SPD is adapted to engage a thermal cycler in a manner so that a DSC can communicate with the operating system or control assembly of said thermal cycler. In one embodiment the DSC communicates by forming an electrical connection with the thermal cycler. In another embodiment the DSC communicates by forming a wireless connection with the thermal cycler.

In some embodiments the structure of the nucleic acid sample preparation device comprises one or more plastics or polymers including but not limited to polyvinyl chloride, polyethylene, polymethyl methacrylate, nylon, polyester, acrylics, silicones, polyurethanes, polyamides, polystyrene, polyethylene terephthalate, polypropylene, acrylonitrile butadiene styrene, polycarbonate, polyvinylidene chloride, bayblend, polymethyl methacrylate, polytetrafluoroethylene, polyetheretherketone, polyetherimide, phenol formaldehydes, urea-formaldehyde, or melamine formaldehyde.

In some embodiments, the nucleic acid sample preparation device can be further surrounded by a sealed pouch, during handling and storage, and prior to being used. In one embodiment the sealed pouch is opague to light. In another embodiment the sealed pouch is substantially airtight. In another embodiment the sealed pouch is heat resistant. In one embodiment the sealed pouch is made out of a plastic. In one embodiment the nucleic acid sample preparation device can be sealed in the pouch with an inert gas. In another embodiment the sealed pouch may also contain a packet of desiccant. The nucleic acid sample preparation device can be disposable.

In one aspect of the invention a kit is supplied comprising a nucleic acid sample preparation device, instructions on how to use said SPD, and a sealed pouch. In one embodiment the nucleic acid sample preparation device and the instructions are supplied in the sealed pouch. In another embodiment the nucleic acid sample preparation device is supplied in the sealed pouch and the instructions are supplied separately or are printed on the sealed pouch. In another embodiment the sealed pouch comprises instructions printed on its surface, either directly or on a label attached to the sealed pouch. In one embodiment the nucleic acid sample preparation device comprises lysis buffer, wash buffer and elution buffer reagents, one or more of which can be present in lyophilized and solvent type format (e.g. dual chamber syringe). In another embodiment the nucleic acid sample preparation device further comprises a reaction mix, which can be supplied in lyophilized and solvent type format, or as a gel. In a further embodiment the reaction mix comprises one or more polynucleotide sequence specific primers or probes.

In some embodiments the SPD processes a sample and delivers it via a collection port to a first device such as a thermal cycler. In some embodiments the device (e.g., thermal cycler) further comprises a light source and photo detector. In one embodiment the device comprises a vacuum for moving a sample through the SPD. In some embodiments the device comprises a vacuum inlet with a vapor bloc component.

In one aspect of the invention a business method is disclosed wherein an SPD comprising at least one empty reagent reservoirs/and or a DSC comprising at least some empty memory is delivered to a distributor. The distributor then loads the at least one empty reagent reservoir of the SPD with distributor supplied reagents and/or encodes the DSC with a distributor supplied computer program. The distributor then distributes the loaded SPD to customers for use.

### Computer Program

In one embodiment, A DSC comprises a computer program that comprises computer executable logic such as computer readable instructions for operating a device, such as a thermal cycler. In some embodiments, a computer program is stored on the computer readable medium of a DSC (e.g., such as a Flash memory card or other mediums disclosed herein).

In some embodiments, a computer program comprises instructions for operating a system comprising an SPD. In one embodiment the computer program comprises instructions for the isolation and/or purification of nucleic acids from a biological sample. The computer readable instructions can comprise instructions for addressing the biological sample under conditions suitable for producing nucleic acids suitable for amplification.

In some embodiments, the computer program comprises one or more instructions to cause the system to perform at least one of the following steps: output an indicator of the placement of an SPD in fluid connection with a collection vessel engaged with a thermal cycler; read a sample label or an SPD label (such as a bar code or a user entered label); load instructions or sample information from a DSC; output directions for a user to input a sample identifier; output directions for a user to load an input of the SPD with a biological sample; output directions for a user to introduce the biological sample into the SPD; output directions for a user to input a reagent (such as custom primers) to the SPD; output directions for a user to cause the biological sample to contact a lysis reagent in the SPD; output directions for a user to fluidly engage an SPD with a thermal cycler; output directions for a user to operate a force member in the apparatus to apply pressure at an interface between a portion of the receiving bay and a portion of the SPD; output directions for a user to pressurize the SPD by engaging a positive pressure device; or output directions for a user to pressurize the SPD by engaging a negative pressure device (such as a vacuum).

In some embodiments, the computer program can include one or more instructions to cause the thermal cycler to perform at least one of the following steps: lyse a biological sample; lyse a biological sample with a lysis reagent; reconstitute a lyophilized pellet of surfactant with liquid to create a lysis reagent solution; heat a biological sample; separate nucleic acids from at least a portion of the biological sample; separate nucleic acids from substantially all of the polymerase chain reaction inhibitors in the biological sample; direct a fluid in the SPD by operating one or more of a positive pressure device, a vacuum, a thermally actuated pump, a pressure actuated valve or a diaphragm valve (such as a MOV that is actuated by pneumatics); contact the processing component with a wash buffer; pump one or more nucleic acids to a collection vessel; heat the sample or partially processed sample (such as nucleic acids), to a temperature of between 4 and 100° C.

In some embodiments, the computer program can include one or more instructions to cause the system to perform at least one of the following steps: combine nucleic acids with a PCR reagent mixture comprising a polymerase enzyme and a plurality of nucleotides; heat a PCR reagent mixture/nucleic acid combination under thermal cycling conditions suitable for creating PCR amplicons from the nucleic acids; contact the nucleic acids or a PCR amplicon thereof with at least one probe that can selectively bind a specific polynucleotide sequence; independently contacting nucleic acids isolated from a biological sample and control nucleic acids (such as a negative control) with a PCR reagent mixture under thermal cycling conditions suitable for independently creating PCR amplicons; contact nucleic acids isolated from a biological sample or a PCR amplicon thereof and control nucleic acids or a PCR amplicon thereof with at least one probe that selectively binds a specific polynucleotide sequence; outputting a determination of the presence of a specific polynucleotide sequence in a biological sample, if a probe detects a specific polynucleotide sequence in nucleic acids isolated from a biological sample or a PCR amplicon thereof; and/or output a determination of a contaminated result if a probe detects a specific polynucleotide sequence in control nucleic acids (such as a negative control, or internal control or standards) or a PCR amplicon thereof.

In some embodiments, the computer program can include one or more instructions to cause the system to perform at least one of the following steps: combine RNA with a reverse transcription reagent mixture comprising a polymerase enzyme and a plurality of nucleotides; heat a reverse transcription reagent mixture/RNA combination under thermal cycling conditions suitable for creating DNA products from the RNA; contact the RNA or DNA products thereof with at least one probe that can selectively bind a specific polynucleotide sequence; independently contacting RNA isolated from a biological sample and control RNA (such as a positive and/or negative control) with a reverse transcription reagent mixture under thermal cycling conditions suitable for independently creating DNA products; contact RNA isolated from a biological sample or a DNA products thereof and control RNA or a DNA products thereof with at least one probe that selectively binds a specific polynucleotide sequence; outputting a determination of the presence of a specific polynucleotide sequence in a biological sample, if a probe detects a specific polynucleotide sequence in RNA isolated from a biological sample or DNA products thereof; and/or output a determination of a contaminated result if a probe detects a specific polynucleotide sequence in control RNA (such as a negative control) or DNA products thereof.

In some embodiments, the computer program can include one or more instructions to cause the system to automatically conduct one or more of the steps of the instructions set forth above. In some embodiments, the computer program includes computer readable instructions thereon for causing a system to isolate and/or analyze a nucleic acid from a sample.

In some embodiments a system comprises a SPD comprising one or more conduits, one or more input ports and optionally, one or more output ports; and an apparatus comprising a receiving bay configured to selectively receive the SPD; at least one heat block adapted to fluidically couple to the SPD in the receiving bay; a detector; and a programmable processor coupled to the detector and the heat pump.

### Sample Processing System

In one aspect a system for carrying out thermal cycling using a fluidic volume is designed, developed, and implemented in a fluidic format. In one embodiment the system comprises a sample processing cartridge (SPD). In one embodiment the SPD comprises one or more delivery units or reagent reservoirs; a housing, comprising a processing component, conduits (including but not limited to a capillary channel, a channel or a channel), a waste chamber or waste port, and a collection port; and, optionally, a collection vessel. In some embodiments one or more nucleic acid processing components comprises a filter capable of binding or capturing one or more nucleic acids, microbeads with nucleic acid binding moieties, or nucleic acid binding moieties bound to a substrate, such as one or more walls of the processing component. In some embodiment the SPD comprises a pre-filter that removes particulate matter (e.g., cells, blood cells, cell components) from the sample prior to nucleic acid capture in the processing component. Optionally, the SPD may comprise one or more waste output ports or waste chambers. In some embodiment the one or more waste output ports or waste chambers comprise an aerosol filter. In some embodiments a waste chamber can be configured to receive and to contain waste, such as fluids and/or particulate matter such as cellular debris. In some embodiments, at least one of the delivery units or reagent reservoirs comprises a wash buffer. In some embodiments the wash buffer has a pH of about 7. In another embodiment the wash buffer has a basic pH, such as between 7 and 11 (including but not limited to, about 7.5, about 8.0, about 8.5, about 9.0, about 9.5, about 10.0 and about 10.5)

In one embodiment the SPD can accommodate sample volumes in the range from about 0.1 μl to about 25 ml, wherein the principal limitation on the lower limit is sensitivity of detection. Exemplary volumes include but are not limited to a range 0.5 ml −10 ml, 0.5 ml to 1 ml, 0.5 ml to 2 ml, 0.5 ml to 3 ml, 0.5 ml to 4 ml, 0.5 ml to 5 ml, 1 ml to 5 ml, 1 ml to 6 ml, 1 ml to 7 ml, 1 ml to 8 ml, 1 ml to 9 ml, 1 ml to 10 ml, 5 ml to 10 ml, 5 ml to 15 ml 5 ml to 20 ml or 5 ml to 25 ml. Still other exemplary volumes include but are not limited to 0.5 ul to 1.5 ml, 1 ul to 500 ul, 100 ul to 1 ml, 500 ul to 1.5 ml.

In some embodiments, chemistry will be optimized and a compatible detection system used to enable two-color multiplex PCR thereby facilitating the use of internal positive controls to check for efficiency of sample prep and proper performance of the associated instrumentation. Due to very small thermal masses and efficient feedback-control based algorithms, it can be possible to perform ultra-fast thermo-cycling. In one embodiment the system comprises thermal cycler with a liquid metal heat block which provides for shorter amplification cycles, while maintaining precise temperature control. In another embodiment the system comprises a conventional metal heat block.

In some embodiments, the SPD comprises a lyophilized or stabilized reaction mix in a delivery unit or a reagent reservoir. In some embodiments the SPD reaction mix comprises all of the reagents necessary to perform a reaction such as polymerase chain reaction (PCR), quantitative polymerase chain reaction (qPCR), nucleic acid sequencing, ligase chain polymerase chain reaction (LCR-PCR), reverse transcription PCR reaction (RT-PCR), single base extension reaction (SBE), multiplex single base extension reaction (MSBE), reverse transcription, or nucleic acid ligation. In one embodiment a reaction mix comprises any number (e.g., 0, 1, 2, or all) of the reagents for performing PCR can be incorporated on the SPD in a lyophilized format. In some embodiments the SPD reaction mix comprises at least one reagent for performing PCR or reverse transcription, including but not limited to DNA polymerase, reverse polymerase, dNTPs, buffer, Mg+, primers, labeled primers, fluorophores, or intercalating dyes. At the time of use, the lyophilized PCR reagents can be reconstituted using, for example, deionized water, which may be stored on the SPD in a dual chamber delivery unit (e.g. a syringe) or a blister format (e.g., in a self-pierceable reservoir). In another embodiment the lyophilized PCR reagents can be reconstituted by delivery of a fluid (such as sterile or deionized water, or a buffer) to the SPD via a sample or reagent input port. In some embodiments, the reconstituted PCR reagents can be aliquoted into, two or more aliquots

In some embodiments the lyophilized reagents can be separated from an internal fluid reservoir or an external fluid input port by a pierceable wall. In one embodiment the wall is formed of a material having a low vapor transmission rate (e.g., Aclar, a metallized (e.g. aluminum) laminate, a plastic, or a foil laminate) that can be ruptured or pierced.

In some embodiments, a fluidic system such as an SPD can include components such as micropumps for moving/mixing liquid drops, microreactors for performing thermally initiated biochemical reactions, and micro valves or microgates to enable control of the liquid pumping operations as well as to isolate regions of the SPD such as the PCR chambers during thermal cycling.

In some embodiments, the SPD can include a filter in fluid communication with the sample inlet valve, the filter being configured to separate at least one component from a sample mixture introduced at the sample inlet. In one embodiment a sample, such as a biological sample can be delivered to an SPD. In one embodiment a biological sample may comprise blood, urine, tears, semen, feces, saliva, sputum, a buccal sample, a lung lavage sample, a vaginal sample, amniotic fluid, a hair bulb, or a tissue sample. In another embodiment the sample is selected from the group consisting of a tissue culture, a plasmid sample, a bacteria culture, a viral culture. In another embodiment the sample may be a water sample, an air sample, a food sample, a drug sample, or any other sample to tested for contamination with a microorganism (such as bacteria or viruses). In another embodiment the sample may comprise one or more eukaryotic, prokaryote or viral nucleic acids. The volume of the sample can be between 1 ul to 5 ml, such as 50 ul to 2.5 ml, more preferably between 100 ul to 1 ml. In one embodiment particulates above a threshold size (e.g., cells) in the fluid sample are removed via filtration and PCR is performed on the filtered fluid sample. In another embodiment cells and/or bacteria in the sample are lysed, such as by chemical, enzymatic, mechanical (e.g. via a maceration blade) or thermal lysis. In one embodiment the SPD comprises at least one lyophilized surfactant. The released nucleic acids can then be processed via fluidic manipulation in the SPD. In some embodiments the nucleic acids are purified for a specific species (e.g. RNA, or DNA). In another embodiment at least one species of nucleic acid is concentrated (e.g. RNA, or DNA). In some embodiments at least one nucleic acid sequence is captured or bound to a substrate in a processing component. In some embodiments the substrate is a filter or a membrane. In some embodiments the substrate comprises hydroxyapitate. In some embodiments the substrate comprises a binding moiety, such as a nucleic acid sequence or a nucleic acid specific antibody or fragment thereof (e.g., Fc, Fab, Scv). In some embodiments the processing component comprises one or more beads (such as ferrous beads or polystyrene beads). In some embodiments at least one nucleic acid sequence is bound to a bead, such as an affinity-microbead. In some embodiments the microbeads can be about 10 microns in size.

In some embodiments, a total amount of beads in the range of a 100,000 to 5 million can be used per SPD for DNA concentration. In some cases, a minimum pressure of 5 psi (e.g., 10 psi, 11 psi, or 15 psi) may be used to concentrate the beads against an inline-filter area of a few square millimeters (such as a pore size of 2-8 microns) in a few minutes (such as 1-3 minutes). This pressure can be generated, for example, by a vacuum, positive pressure pump or by injecting air (e.g., 1-3 mL) into SPD. Thus, it should be made clear that the pressure used can be positive pressure or negative pressure. In some embodiments, a one-way duckbill valve at a Luer or similar means for inlet can be used to minimize or prevent air pressure from escaping or entering through an inlet. In another embodiment an SPD comprises one or more membrane valve ports. In some embodiment the membrane is a resalable airtight/water tight polymer (such as Sifel). In one embodiment membrane valves seal the input and/or output ports of the SPD.

### Thermal Cycler

An SPD of the device can be configured to be operably linked to any PCR machine. For example, by use of slide and groove, male/female ports, spring or snap-on fittings, or any means of attachment conventional in the art, a SPD can be fitted to a PCR machine. Furthermore, the SPD can comprise a DSC which has data or computer executable logic provided that corresponds to a particular PCR device or PCR devices. In addition, as noted herein, a DSC can comprise computer executable logic for operating a particular assay or diagnostic, as well as for a particular sample or samples.

In some embodiments, a SPD operably linked to a PCR machine can produce a readout or output (e.g., detection of a target molecule) in less than about 45 minutes, less than about 40 minutes, less than about 39 minutes, less than about 38 minutes, less than about 37 minutes, less than about 36 minutes, less than about 35 minutes, less than about 34 minutes, less than about 33 minutes, less than about 32 minutes, less than about 31 minutes, less than about 30 minutes, less than about 29 minutes, less than about 28 minutes, less than about 27 minutes, less than about 26 minutes, less than about 25 minutes, less than about 24 minutes, less than about 23 minutes, less than about 22 minutes, less than about 21 minutes, less than about 20 minutes, less than about 19 minutes, less than about 18 minutes, less than about 17 minutes, less than about 16 minutes or less than about 15 minutes. In various embodiments, a readout or output is provided in about 15-20 minutes, about 20-30 minutes, about 25-35 minutes, about 30 to 45 minutes. In one embodiment a readout or output is provided in about 30 to 35 minutes.

In one aspect of the invention an apparatus comprising a thermal cycler for cycling the nucleic acids and optionally, a reaction mix delivered from an SPD is provided. In some embodiments the thermal cycler further comprises an optical assembly for detecting signal from a reaction mix and control means for controlling the operation of the thermal cycler and the optical assembly.

In some embodiments the thermal cycler employs a peltier device. In some embodiments the thermal cycler employs a conventional metal heat block (e.g. a solid metal heat block). In another embodiment the thermal cycler employs heated air (e.g. an air cycler). In another embodiment the thermal cycler employs a heat block comprising a liquid composition (such as a liquid metal or a thermally conductive fluid) to rapidly cycle the temperatures in a reaction mixture. The use of a liquid metal provides two main advantages. First, metal has high thermal conductivity, providing rapid heat transfer. Second, liquid provides tighter contact between the thermally conductive material and the SPD or collection vessel, providing more uniform heat transfer. The combination of rapid temperature ramp rates and uniformity of temperature decreases non-specific hybridization and significantly increases the specificity (e.g., signal-to-noise ratio) of amplification in PCR within individual reaction mixtures as well as across multiple reaction mixtures located in the same heat block. In another embodiment, a reaction mixture emits substantially all of a signal generated therein out through a discrete portion of a collection vessel, for example, the top or a cap, whereby the emitted light can be collected by the optical assembly. In yet another embodiment a light detector detects substantially all of the light emitted from a collection vessel. In certain embodiments the liquid metal or collection vessel is highly reflective and reflects light transmitted through the walls of a transparent location on an collection vessel back into the collection vessel. In this way, a greater proportion of a light signal generated inside the collection vessel is emitted from a discrete portion of the collection vessel, whereby it can be collected by the optical assembly. The ability to collect more light from the reaction means that less expensive optics can be employed in the device, thereby decreasing the cost. Furthermore, collecting light from a discrete location of an collection vessel eliminates the necessity of removing an collection vessel from the heat block when performing real time PCR. Thus, the configuration of the heat block allows rapid ramp times and uniform temperatures, and the collection of reflected light from a surface of a collection vessel by the optical assembly without removing a collection vessel from the heat block, allows real time PCR to proceed more quickly. Accordingly, the apparatus of this invention is particularly adapted for performing PCR (polymerase chain reaction), reverse transcription PCR and real time PCR. Thermal cyclers comprising the liquid metal heat block will perform PCR faster and more cheaply than devices presently available on the market. In one embodiment a thermal cycler comprising a heat block comprising a liquid composition is powered by a battery. In another embodiment a thermal cycler comprising a heat block comprising a liquid composition is powered by a AC or DC current.

The use of a liquid composition, such as a liquid metal or a thermally conductive fluid, as a heating and cooling medium for a heating block, results in a more uniform heat transfer and more rapid heating and cooling cycles than solid metal heat blocks. In embodiments where a liquid metal heat block is used as a thermal cycler, the faster heat ramping, and superior thermal uniformity lead to lower error rates by DNA polymerases than when used in conventional thermal cyclers. This is due to the decreased time in which the PCR sample spends at sub-optimal temperatures. Further, the error rates are decreased during long amplifications, SNP identification and sequencing reactions, because of the enhanced thermal uniformity. In one embodiment the liquid metal thermocycler disclosed in co-pending application 2008/0003649, filed May 17, 2007 (which is herein incorporated by reference in its entirety) is used with the SPD disclosed above.

### Control Assembly

In various embodiments a control assembly is operatively linked to a thermal cycler of the invention. Such a control assembly, for example, comprises a programmable computer comprising, computer executable logic that functions to operate any aspect of the devices, methods and/or systems of the invention. For example, the control assembly can turn on/off motors, fans, heating components, stir bars, continuous flow devices, optical assemblies, positive pressure pumps, or vacuum pumps. The control assembly can be programmed to automatically process samples, run multiple PCR cycles, obtain measurements, digitize measurements into data, convert data into charts/graphs and report.

Computers for controlling instrumentation, recording signals, processing and analyzing signals or data can be any of a personal computer (PC), digital computers, a microprocessor based computer, a portable computer, or other type of processing device. Generally, a computer comprises a central processing unit, a storage or memory unit that can record and read information and programs using machine-readable storage media, a communication terminal such as a wired communication device or a wireless communication device, an output device such as a display terminal, and an input device such as a keyboard. The display terminal can be a touch screen display, in which case it can function as both a display device and an input device. Different and/or additional input devices can be present such as a pointing device, such as a mouse or a joystick, and different or additional output devices can be present such as an enunciator, for example a speaker, a second display, or a printer. The computer can run any one of a variety of operating systems, such as for example, any one of several versions of Windows, or of MacOS, or of Unix, or of Linux.

In some embodiments, the control assembly executes the necessary computer programs to digitize the signals detected and measured from one or more SPDs and processes the data into a readable form (e.g., table, chart, grid, graph or other output known in the art). Such a form can be displayed or recorded electronically or provided in a paper format. In other embodiments the control assembly executes programs present on the DSC of an SPD. In some embodiments the DSC comprises at least one computer program (e.g., software), including but not limited to testing programs (e.g., programs to analyze melting curve data, RT-PCR data analysis), calibration programs, verification programs, software updates to the system, or other programs. In one embodiment the SPD comprises software that links the SPD directly to patient specific data. In another embodiment, data obtained by analysis with the SPD are delivered to a health or research professional. In some embodiments the delivery is automatic. In some embodiments the software encrypts the data to insure its security.

In some embodiments, the control assembly controls circuitry linked to the thermal elements so as to regulate/control cycles temperatures of a thermal cycler of the invention.

In another embodiment, the control assembly generates the sampling strobes of the optical assembly, the rate of which is programmed to run automatically. Of course it will be apparent, that such timing can be adjusted for particular light sources and the corresponding detector in order to optimize signal detection and measurement (e.g., fluorescence).

In another embodiment an apparatus comprising a control assembly further comprises a means for moving an SPD into an opening in a receiving bay of a heat block comprising a liquid composition. In another embodiment said means could be a robotic system comprising motors, pulleys, clamps and other structures necessary for moving an SPD.

Sample preparation station. In some aspects of the invention, the devices/systems of the invention are operatively linked to a robotics sample preparation and/or sample processing unit. For example, a control assembly can provide a program to operate automated collection of samples, and input into one or more SPDs, optionally adding additional reagents to one or more SPDs, processing the nucleic acids in said SPDs, performing thermal cycling said nucleic acids (such as PCR, real-time PCR, reverse transcription, ligation, hybridization or enzyme digestion), analyzing said samples (such as by detecting a fluorescent dye or probe), and optionally recovering the processed nucleic acids. In some embodiments, the sample preparation can be in a continuous flow PCR system described herein or in a non-continuous system.

### Methods of Nucleic Acid Analysis

In one aspect a method is disclosed for the isolation and/or analysis of a nucleic acid present in a sample. In one embodiment a method for isolation and/or analysis of a nucleic acid present in a sample comprises contacting an SPD with a sample (such as a biological sample). Wherein, the biological sample comprises at least one nucleic acid sequence, such as RNA or DNA. In one embodiment the sample is lysed and at least one nucleic acid sequence is captured in a processing component (such as by a filter or microbead). In some embodiments the processing component captures substantially more DNA nucleic acids than RNA nucleic acids. In some embodiments the processing component captures specifically DNA nucleic acids. In some embodiments the processing component captures substantially more RNA nucleic acids than DNA nucleic acids. In some embodiments the processing component captures specifically RNA nucleic acids. In some embodiments the at least one captured nucleic is washed by a wash buffer.

In some embodiments, a method of isolation and/or analysis of a nucleic acid comprises one or more of the following steps: engaging an SPD with a device (such as thermal cycler); and delivering nucleic acids and or a reaction mix to a collection vessel.

In some embodiments, a method of isolation and/or analysis of a nucleic acid comprises reading a computer-readable label on the SPD or a label on the biological sample. For example, the label can include an optically readable code, such as a bar code, Dotcode (such as Dotcode-128) a radio frequency tag (RFID tag), one or more computer-readable characters or a smartcard chip.

In some embodiments, a method of isolation and/or analysis of a nucleic acid comprises introducing a crude sample (such as a crude biological sample) into a SPD and separating a fractional biological sample from the crude biological sample in the SPC, e.g., using a filter in the cartridge, or the fractional biological sample can be separated from a crude biological sample prior to introducing the biological sample into the SPD. In some embodiments, the method comprises lysing a biological sample, for example, using heat, or a lysis reagent. In some embodiments, wherein the SPD comprises one or more lyophilized pellets of lysis reagent, the method comprises reconstituting the lyophilized pellet of surfactant with liquid to create a lysis reagent solution.

In some embodiments, a method of isolation and/or analysis of a nucleic acid comprises one or more of the following: heating the biological sample in a collection vessel, pressurizing a biological sample in the SPD at a pressure differential compared to ambient pressure of between about 20 kilopascals and 200 kilopascals, or in some embodiments between about 70 kilopascals and 110 kilopascals. In some embodiments the pressure is positive pressure. In some embodiments the pressure is negative pressure.

In some embodiments, a method of isolation and/or analysis of a nucleic acid comprises pumping fluids (such as a sample or reagents) in a channel or a capillary using diaphragm valves. In one embodiment the diaphragm valve is a MOV valve, which can be linked in a series of three or more to pump fluids through a channel or a capillary (U.S. Pat. No. 6,551,839; U.S. patent application Ser. No. 11/229,065; U.S. Pat. No. 6,190,616; U.S. Pat. No. 6,423,536; U.S. application Ser. No. 09/770,412; U.S. Pat. No. 6,870,185; U.S. application Ser. No. 10/125,045; U.S. application Ser. No. 10/540,658; U.S. patent application Ser. No. 10/750,533; U.S. patent application Ser. No. 11/138,018; all of which are herein incorporated by reference in their entirety)

In some embodiments, a portion of the nucleic acids isolated in the SPD can include at least one polymerase chain reaction inhibitor selected from the group consisting of hemoglobin, peptides, fecal compounds, humic acids, mucosal compounds, DNA binding proteins, or a sacoharide. In some embodiments, a method further comprises separating at least one nucleic acid from substantially all of the polymerase chain reaction inhibitors in the biological sample.

In some embodiments, a method of isolation and/or analysis of a nucleic acid comprises one or more of the following: directing a fluid in the SPD by operating a vacuum pump, a positive pressure device, a thermally actuated pump or a thermally actuated valve; contacting the processing component with a wash buffer; contacting the processing component with a release buffer to create a released polynucleotide sample (for example, in some embodiments, the release buffer can have a volume of less than about 5 mls, the release buffer can include a chelating agent, and/or the release buffer can have a pH of at least about 10); and/or contacting the released polynucleotide sample with a neutralization buffer to create a neutralized polynucleotide sample.

In some embodiments, a method of isolation and/or analysis of a nucleic acid comprises one or more of the following: contacting a nucleic acid sequence with a PCR reagent mixture comprising a polymerase enzyme and a plurality of nucleotides an optionally a fluorescent oligonucleotide probe. In some embodiments, the PCR reagent mixture can be in the form of one or more lyophilized pellets, and the method can comprise reconstituting the PCR pellet with liquid to create a PCR reagent mixture solution; heating the PCR reagent mixture and a nucleic acid under thermal cycling conditions suitable for creating PCR amplicons from the nucleic acid; contacting the nucleic acid or a PCR amplicon thereof with at least one probe that can selectively bind a specific nucleic acid. In some embodiments the method can comprise independently contacting nucleic acids isolated from a sample and control nucleic acids (such as a negative control) with a PCR reagent mixture under thermal cycling conditions suitable for independently creating PCR amplicons; contact nucleic acids isolated from a sample or a PCR amplicon thereof and control nucleic acids or a PCR amplicon thereof with at least one probe that selectively binds a specific polynucleotide sequence.

In some embodiments the method can comprise one or more of the following: determining the presence of a specific polynucleotide sequence in a sample, if a probe binds a specific polynucleotide sequence in nucleic acids isolated from a sample or a PCR amplicon thereof; and/or determining if the results are contaminated when a probe detects a specific polynucleotide sequence in control nucleic acids (such as a negative control) or a PCR amplicon thereof.

In one aspect a system comprising an SPD and a thermal cycler (such as a thermal cycler comprising a liquid metal, thermally conductive fluid, air cycler, or conventional heat block) can be used for methods, including but not limited to, disease diagnosis, drug screening, genotyping individuals, phylogenetic classification, environmental surveillance, parental and forensic identification amongst other uses. Further, nucleic acids can be obtained from any source for analysis in a system comprising an SPD and a thermal cycler using a liquid metal or a thermally conductive fluid heat block. For example, the source can be a test sample such as a biological and/or environmental samples. Biological samples may be derived from human, other animals, or plants, housing fluid, solid tissue samples, tissue cultures or cells derived there from and the progeny thereof, sections or smears prepared from any of these sources, or any other samples suspected to contain the target nucleic acids. Exemplary biological samples are housing fluids including but not limited to blood, urine, spinal fluid, cerebrospinal fluid, sinovial fluid, ammoniac fluid, semen, and saliva. Other types of biological sample may include food products and ingredients such as vegetables, dairy items, meat, meat by-products, and waste. Environmental samples are derived from environmental material including but not limited to soil, water, sewage, cosmetic, agricultural, industrial samples, air filter samples, and air conditioning samples.

In one embodiment a system comprising an SPD and a thermal cycler further comprises a detection system. For example said thermal cycler can be used for polymerase chain reaction (PCR), quantitative polymerase chain reaction (qPCR), nucleic acid sequencing, ligase chain polymerase chain reaction (LCR-PCR), reverse transcription PCR reaction (RT-PCR), single base extension reaction (SBE), multiplex single base extension reaction (MSBE), reverse transcription, and nucleic acid ligation. In some embodiments the detection system may comprise a light source and/or a light detector. In some embodiments the thermal cycler comprises a liquid metal or a thermally conductive fluid heat block. In some embodiments the thermal cycler comprises a conventional solid metal heat block.

A thermal cycler comprises a liquid metal or a thermally conductive fluid heat block allows one to perform PCR with increased speed and specificity, particularly in the context of real time PCR. The use of a composition with high thermal conductivity, such as a liquid metal, allows one to perform temperature ramping (both up and down) much faster than traditional PCR. This not only increases the potential speed at which one can carry out PCR, but it also increases the specificity of PCR by decreasing the incidence of non-specific hybridization of primers. Furthermore, in the context of real time PCR, measuring signal from a discrete portion of the test receiving bay, such as the top, relieves one of the need to remove an SPD from the heating composition for measurement. This also preserves temperature control and allows measurements to be made in real time with the heating cycles. The use of a reflecting material that prevents escape of signal except from the discrete location allows less sensitive detectors to be used as more light can be collected for measurement.

PCR reaction conditions typically comprise either two or three step cycles. Two step cycles have a denaturation step followed by a hybridization/elongation step. Three step cycles comprise a denaturation step followed by a hybridization step during which the primer hybridizes to the strands of DNA, followed by a separate elongation step. The polymerase reactions are incubated under conditions in which the primers hybridize to the target sequences and are extended by a polymerase. The amplification reaction cycle conditions are selected so that the primers hybridize specifically to the target sequence and are extended.

Successful PCR amplification requires high yield, high selectivity, and a controlled reaction rate at each step. Yield, selectivity, and reaction rate generally depend on the temperature, and optimal temperatures depend on the composition and length of the polynucleotide, enzymes and other components in the reaction system. In addition, different temperatures may be optimal for different steps. Optimal reaction conditions may vary, depending on the target sequence and the composition of the primer. Thermal cyclers may be programmed by selecting temperatures to be maintained, time durations for each cycle, number of cycles, rate of temperature change and the like.

Primers for amplification reactions can be designed according to known algorithms. For example, algorithms implemented in commercially available or custom software can be used to design primers for amplifying desired target sequences. Typically, primers can range are from least 12 bases, more often 15, 18, or 20 bases in length but can range up to 50+ bases in length. Primers are typically designed so that all of the primers participating in a particular reaction have melting temperatures that are within at least 5° C., and more typically within 2° C. of each other. Primers are further designed to avoid priming on themselves or each other. Primer concentration should be sufficient to bind to the amount of target sequences that are amplified so as to provide an accurate assessment of the quantity of amplified sequence. Those of skill in the art will recognize that the amount of concentration of primer will vary according to the binding affinity of the primers as well as the quantity of sequence to be bound. Typical primer concentrations will range from 0.01 uM to 0.5 uM.

In one embodiment, a liquid metal or thermally conductive fluid heating block may be used for PCR, either as part of a thermal cycler or as a heat block used to maintain a single temperature. In a typical PCR cycle, a sample comprising a DNA polynucleotide and a PCR reaction cocktail is denatured by treatment in a liquid metal or thermally conductive fluid heat block at about 90-98° C. for 10-90 seconds. The denatured polynucleotide is then hybridized to oligonucleotide primers by treatment in a liquid metal or thermally conductive fluid heat block at a temperature of about 30-65° C. for 1-2 minutes. Chain extension then occurs by the action of a DNA polymerase on the polynucleotide annealed to the oligonucleotide primer. This reaction occurs at a temperature of about 70-75° C. for 30 seconds to 5 minutes in the liquid metal or thermally conductive fluid heat block. Any desired number of PCR cycles may be carried out depending on variables including but not limited to the amount of the initial DNA polynucleotide, the length of the desired product and primer stringency.

In another embodiment, the PCR cycle comprises denaturation of the DNA polynucleotide at a temperature of 94° degree C. for about 1 minute. The hybridization of the oligonucleotide to the denatured polynucleotide occurs at a temperature of about 37°-65° C. for about one minute. The polymerase reaction is carried out for about one minute at about 72.degree. C. All reactions will be carried out in an SPD which is inserted into a receiving bay in a liquid metal or thermally conductive fluid heat block. About 30 PCR cycles are performed. The above temperature ranges and the other numbers are not intended to limit the scope of the invention. These ranges are dependant on other factors such as the type of enzyme, the type of container or plate, the type of biological sample, the size of samples, etc. One of ordinary skill in the art will recognize that the temperatures, time durations and cycle number can readily be modified as necessary.

### Reverse Transcription PCR

Revere transcription refers to the process by which mRNA is copied to cDNA by a reverse transcriptase (such as Moloney murine leukemia virus (MMLV) transcriptase Avian myeloblastosis virus (AMV) transcriptase or a variant thereof) composed using an oligo dT primer or a random oligomers (such as a random hexamer or octamer). In real-time PCR, a reverse transcriptase that has an endo H activity is typically used. This removes the mRNA allowing the second strand of DNA to be formed. Reverse transcription typically occurs as a single step before PCR. In one embodiment the RT reaction is performed in a liquid metal or thermally conductive fluid heat block by incubating an RNA sample a transcriptase the necessary buffers and components for about an hour at about 37° C., followed by incubation for about 15 minutes at about 45° C. followed by incubation at about 95° C. The cDNA product is then removed and used as a template for PCR. In an alternative embodiment the RT step is followed sequentially by the PCR step, for example in a one-step PCR protocol. In this embodiment all of the reaction components are present in the SPD for the RT step and the PCR step. However, the DNA polymerase is blocked from activity until it is activated by an extended incubation at 95° C. for 5-10 minutes. In one embodiment the DNA polymerase is blocked from activity by the presence of a blocking antihousing that is permanently inactivated during the 95° C. incubation step.

### Real Time PCR

In molecular biology, real-time polymerase chain reaction, also called quantitative real time polymerase chain reaction (QRT-PCR) or kinetic polymerase chain reaction, is used to simultaneously quantify and amplify a specific part of a given DNA molecule. It is used to determine whether or not a specific sequence is present in the sample; and if it is present, the number of copies in the sample. It is the real-time version of quantitative polymerase chain reaction (Q-PCR), itself a modification of polymerase chain reaction.

The procedure follows the general pattern of polymerase chain reaction, but the DNA is quantified after each round of amplification; this is the “real-time” aspect of it. In one embodiment the DNA is quantified by the use of fluorescent dyes that intercalate with double-strand DNA. In an alternative embodiment modified DNA oligonucleotide probes that fluoresce when hybridized with a complementary DNA are used to quantify the DNA.

In another embodiment real-time polymerase chain reaction is combined with reverse transcription polymerase chain reaction to quantify low abundance messenger RNA (mRNA), enabling a researcher to quantify relative gene expression at a particular time, or in a particular cell or tissue type.

In certain embodiments, the amplified products are directly visualized with detectable label such as a fluorescent DNA-binding dye. In one embodiment the amplified products are quantified using an intercalating dye, including but not limited to SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin. For example, a DNA binding dye such as SYBR Green binds all double stranded (ds) DNA and an increase in fluorescence intensity is measured, thus allowing initial concentrations to be determined. A standard PCR reaction cocktail is prepared as usual, with the addition of fluorescent dsDNA dye and added to a sample. The reaction is then run in a liquid metal heatblock thermal cycler, and after each cycle, the levels of fluorescence are measured with a camera. The dye fluoresces much more strongly when bound to the dsDNA (i.e. PCR product). Because the amount of the dye intercalated into the double-stranded DNA molecules is typically proportional to the amount of the amplified DNA products, one can conveniently determine the amount of the amplified products by quantifying the fluorescence of the intercalated dye using the optical systems of the present invention or other suitable instrument in the art. When referenced to a standard dilution, the dsDNA concentration in the PCR can be determined. In some embodiments the results obtained for a sequence of interest may be normalized against a stably expressed gene (“housekeeping gene”) such as actin, GAPDH, or 18 s rRNA.

In various embodiments, labels/dyes detected by systems or devices of the invention. The term “Label” or “dye” refers to any substance which is capable of producing a signal that is detectable by visual or instrumental means. Various labels suitable for use in the present invention include labels which produce signals through either chemical or physical means, such as flourescent dyes, chromophores, electrochemical moieties, enzymes, radioactive moieties, phosphorescent groups, fluorescent moieties, chemiluminescent moieties, or quantum dots, or more particularly, radiolabels, fluorophore-labels, quantum dot-labels, chromophore-labels, enzyme-labels, affinity ligand-labels, electromagnetic spin labels, heavy atom labels, probes labeled with nanoparticle light scattering labels or other nanoparticles, fluorescein isotbiocyanate (FITC), TRITC, rhodamine, tetramethylrhodamine, R-phycoerythrin, Cy-3, Cy-5, Cy-7, Texas Red, Phar-Red, allophycocyanin (APC), probes such as Taqman probes, TaqMan Tamara probes, TaqMan MGB probes or Lion probes (Biotools), flourescent dyes such as Sybr Green I, Sybr Green II, Sybr gold, CellTracker Green, 7-AAD, ethidium homodimer I, ethidium homodimer II, ethidium homodimer III or ethidium bromide, epitope tags such as the FLAG or HA epitope, and enzyme tags such as alkaline phosphatase, horseradish peroxidase, I²-galactosidase, alkaline phosphatase, □-galactosidase, or acetylcholinesterase and hapten conjugates such as digoxigenin or dinitrophenyl, or members of a binding pair that are capable of forming complexes such as streptavidin/biotin, avidintbiotin or an antigen/antihousing complex including, for example, rabbit IgG and anti-rabbit IgG; fluorophores such as umbelliferone, fluorescein, fluorescein isothiocyanate, rhodamine, tetramethyl rhodamine, eosin, green fluorescent protein, erythrosin, coumarin, methyl coumarin, pyrene, malachite green, stilbene, lucifer yellow, Cascade Blue, dichlorotriazinylamine fluorescein, dansyl chloride, phycoerythrin, fluorescent lanthanide complexes such as those including Europium and Terbium, Cy3, Cy5, molecular beacons and fluorescent derivatives thereof, a luminescent material such as luminol; light scattering or plasmon resonant materials such as gold or silver particles or quantum dots; or radioactive material including ¹⁴C, ¹²³I, ¹²⁴I, ¹²⁵I, ¹³¹I, Tc99m, ³⁵S or ³H; or spherical shells, and probes labeled with any other signal generating label known to those of skill in the art. For example, detectable molecules include but are not limited to fluorophores as well as others known in the art as described, for example, in Principles of Fluorescence Spectroscopy, Joseph R. Lakowicz (Editor), Plenum Pub Corp, 2nd edition (July 1999) and the 6^{th} Edition of the Molecular Probes Handbook by Richard P. Hoagland.

Intercalating dyes are detected using the devices of the invention include but are note limited to phenanthridines and acridines (e.g., ethidium bromide, propidium iodide, hexidium iodide, dihydroethidium, ethidium homodimer-1 and -2, ethidium monoazide, and ACMA); some minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS751, and hydroxystilbamidine. All of the aforementioned nucleic acid stains are commercially available from suppliers such as Molecular Probes, Inc.

Still other examples of nucleic acid stains include the following dyes from Molecular Probes: cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -83, -84, -85 (orange), SYTO-64, -17, -59, -61, -62, -60, -63 (red). Other detectable markers include chemiluminescent and chromogenic molecules, optical or electron density markers, etc.

In some embodiments, labels comprise semiconductor nanocrystals such as quantum dots (i.e., Qdots), described in U.S. Pat. No. 6,207,392. Qdots are commercially available from Quantum Dot Corporation. The semiconductor nanocrystals useful in the practice of the invention include nanocrystals of Group II-VI semiconductors such as MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, and HgTe as well as mixed compositions thereof; as well as nanocrystals of Group III-V semiconductors such as GaAs, InGaAs, InP, and InAs and mixed compositions thereof. The use of Group IV semiconductors such as germanium or silicon, or the use of organic semiconductors, may also be feasible under certain conditions. The semiconductor nanocrystals may also include alloys comprising two or more semiconductors selected from the group consisting of the above Group III-V compounds, Group II-VI compounds, Group IV elements, and combinations of same.

In addition to various kinds of fluorescent DNA-binding dye, other luminescent labels such as sequence specific probes can be employed in the amplification reaction to facilitate the detection and quantification of the amplified product. Probe based quantitative amplification relies on the sequence-specific detection of a desired amplified product. Unlike the dye-based quantitative methods, it utilizes a luminescent, target-specific probe (e.g., TaqMan® probes) resulting in increased specificity and sensitivity. Methods for performing probe-based quantitative amplification are well established in the art and are taught in U.S. Pat. No. 5,210,015.

In another embodiment fluorescent oligonucleotide probes are used to quantify the DNA. Fluorescent oligonucleotides (primers or probes) containing base-linked or terminally-linked fluorophores and quenchers are well-known in the art. They can be obtained, for example, from Life Technologies (Gaithersburg, Md.), Sigma-Genosys (The Woodlands, Tex.), Genset Corp. (La Jolla, Calif.), or Synthetic Genetics (San Diego, Calif.). Base-linked fluors are incorporated into the oligonucleotides by post-synthesis modification of oligonucleotides that are synthesized with reactive groups linked to bases. One of skill in the art will recognize that a large number of different fluorophores are available, including from commercial sources such as Molecular Probes, Eugene, Oreg. and other fluorophores are known to those of skill in the art. Useful fluorophores include: fluorescein, fluorescein isothiocyanate (FITC), carboxy tetrachloro fluorescein (TET), NHS-fluorescein, 5 and/or 6-carboxy fluorescein (FAM), 5-(or 6-) iodoacetamidofluorescein, 5-{[2 (and 3)-5-(Acetylmercapto)-succinyl]amino}fluorescein (SAMSA-fluorescein), and other fluorescein derivatives, rhodamine, Lissamine rhodamine B sulfonyl chloride, Texas red sulfonyl chloride, 5 and/or 6 carboxy rhodamine (ROX) and other rhodamine derivatives, coumarin, 7-amino-methyl-coumarin, 7-Amino-4-methylcoumarin-3-acetic acid (AMCA), and other coumarin derivatives, BODIPY™ fluorophores, Cascade Blue™ fluorophores such as 8-methoxypyrene-1,3,6-trisulfonic acid trisodium salt, Lucifer yellow fluorophores such as 3,6-Disulfonate-4-amino-naphthalimide, phycobiliproteins derivatives, Alexa fluor dyes (available from Molecular Probes, Eugene, Oreg.) and other fluorophores known to those of skill in the art. For a general listing of useful fluorophores, see also Hermanson, G. T., BIOCONJUGATE TECHNIQUES (Academic Press, San Diego, 1996).

Embodiments using fluorescent reporter probes produce accurate and reliable results. Sequence specific RNA or DNA based probes are used to specifically quantify the probe sequence and not all double stranded DNA. This also allows for multiplexing—assaying for several genes in the same reaction by using specific probes with different-colored labels.

In one embodiment real time PCR is carried out in a thermal cycler comprising a the liquid metal or thermally conductive fluid heat block comprising a liquid composition. In another embodiment real time PCR is carried out in a thermal cycler comprising an air cycler. In another embodiment real time PCR is carried out in a thermal cycler comprising a convention metal heat block. In one embodiment, the thermal cycler further comprises an optical assembly. In another embodiment the liquid metal or thermally conductive fluid heat block rapidly and uniformly modulates the temperature of one or more samples contained within an SPD to allow detection of amplification products in real time. In another embodiment the detection is via a non-specific nucleic acid label such as an intercalating dye, wherein the signal index, or the positive fluorescence intensity signal generated by a specific amplification product is at least 3 times the fluorescence intensity generated by a PCR control sample, such as about 3.5, 4.5, 5, 5.5, 6, 6.5, 7, 7.5, 8, 8.5, 9, 9.5, 10, 10.5, or 11. In an embodiment the thermal cycler may modulate the sample temperature by more than 10° C. per second, such as 10.5° C. per second.

In one embodiment an RNA based probe with a fluorescent reporter and a quencher held in adjacent positions is used. The close proximity of the reporter to the quencher prevents its fluorescence, it is only after the breakdown of the probe that the fluorescence is detected. This process depends on the 5′ to 3′ exonuclease activity of the polymerase used in the PCR reaction cocktail.

Typically, the reaction is prepared as usual, with the addition of the sequence specific labeled probe the reaction commences. After denaturation of the DNA the labeled probe is able to bind to its complementary sequence in the region of interest of the template DNA. When the PCR reaction is heated to the proper extension temperature by the liquid metal or thermally conductive fluid block, the polymerase is activated and DNA extension proceeds. As the polymerization continues it reaches the labeled probe bound to the complementary sequence of DNA. The polymerase breaks the RNA probe into separate nucleotides, and separates the fluorescent reporter from the quencher. This results in an increase in fluorescence as detected by the optical assembly. As PCR progresses more and more of the fluorescent reporter is liberated from its quencher, resulting in a well defined geometric increase in fluorescence. This allows accurate determination of the final, and initial, quantities of DNA.

### Diagnostic Use

In various applications, devices of the invention can be utilized for in vitro diagnostic uses, such as detecting infectious or pathogenic agents. In one embodiment, an SPD is used to prepare nucleic acids from a sample to detect a pathogen or infectious agent, such as, without any limitation, bacteria, yeast, fungi, virus, eukaryotic parasites, etc; infectious agent including influenza viruses A, B or C, parainfluenza virus, adenovirus, rhinovirus, coronavirus, hepatitis viruses A, B, C, D, or E, HIV, enterovirus, papillomavirus, coxsackievirus, herpes simplex virus, or Epstein-Barr virus; bacteria including Mycobacterium, Streptococcus virus (such as a member of group A, B, C, or D), Salmonella, Shigella, Staphylcococcus, Neisseria, Pseudomonads, Clostridium, or E. coli. It will be apparent to one of skill in the art that PCR, sequencing reactions and related processes are readily adapted to the devices of the invention for use to detect any infectious agents.

One advantage of the devices of the invention is the capability to perform fast nucleic acid isolation and preparation for PCR, which provides relatively faster times for diagnostic purposes. For some applications (e.g., detection of biothreat agents, intra-operative diagnostic testing), rapid diagnosis is a benefit. In some embodiments the SPD is coupled to a rapid PCR thermocycler (such as a liquid metal thermocyler) to reduce processing time.

Furthermore, fast PCR processes can be conducted by coupling a fast thermal cyler with reagents known in the art to facilitate faster results, in both amplification and time required to produce a detectable signal. Such reagents are known in the art, such as disclosed in U.S. Patent Application No. 2005/0164219.

For example, specialized labeled primers can provide signal generation that is nearly instantaneous. (2005/0164219, which is herein incorporated by reference in its entirety). A reaction that is extended in the previous cycle undergoes an internal rearrangement, and as soon as the extension temperature is reached, the signal is generated. In a standard PCR reaction with slow cycling conditions, this signal generation difference is not significant. However, when the extension times are reduced in rapid PCR, this feature becomes an advantage and translates into fast PCR. For example, with a liquid metal thermal cycler a single-copy bacterial sequences may be detected in less than 15 minutes after nucleic acid isolation is complete. One example is the rapid detection of low levels of Bacillus spp using Scorpions primers and a fast PCR machine. Furthermore, depending on the amount of input DNA an infectious agent may be detected in less than 10 minutes, and even low levels could be detected in less than 14 minutes. The SPD of the invention can be configured to be used with any PCR machine.

Given the benefits of a self-contained SPD that is transportable and storable without the requirement of cold storage, in some aspects of the invention, an SPD can.

In one aspect a method for rapid detection of a pathogen is disclosed. In one embodiment a biological or environmental sample is processed with an SPD, which delivers at least one nucleic acid sequence and a reagent mix to a thermal cycler. In one embodiment the thermal cycler is a rapid thermal cycler (such as an air cycler or a liquid metal thermal cycler). In one embodiment the thermal cycler comprises an optical detector. In some embodiments an SPD and a rapid thermal cycler are used to detect the presence of a pathogen in less than one hour, such as less than 45 minutes, 30 minutes 25 minutes, 20 minutes, 15 minutes or 10 minutes.

Some aspects of the invention A method of distributing a sample preparation device (SPD) to a distributor; wherein said distributor provides one or more said SPD, wherein each of said SPD is configured to comprise all necessary reagents for isolation of a target compound; and wherein said SPD is configured for storage or transport by said distributor. For example, in one embodiment of the invention, a SPD is self-contained with all the reagents, enzymes, buffers and solvents necessary to conduct an assay (e.g., PCR). Thus a distributor can sell, transport, store and otherwise disseminate SPD(s) without the need for cold storage, and as one unit. Alternatively, compartments containing the various reagents, solvents, enzymes, or buffers can be distributed separately and configured to a housing as described herein.

In yet other embodiments, the SPD so distributed, sold, transported or stored, also comprise a DSC which is configured for one or more particular assays, use with one or more particular machines (e.g., PCR machines) or use detection of one or more particular target molecule (e.g., nucleic acids from pathogens). A method of distributing a sample preparation device (SPD) to a distributor; wherein said SPD comprises: all necessary reagents for processing a sample and obtaining a target compound; a data storage component (DSC) comprising computer executable logic designed to store and analyze data derived from said processing; wherein said computer executable logic alternatively further functions to provide instructions for operation of a PCR device configured to be operably coupled to said SPD.

In another aspect of the invention, a sample preparation device cartridge comprising: a first compartment adapted to receive a sample containing an analyte; a second compartment containing at least one reagent for performing a reaction on the analyte; an outlet; means for delivering the analyte and the at least one reagent from the outlet; and a data storage component comprising, in electronic form, a readable program for performing a reaction protocol on the analyte using the at least one reagent. In one embodiment, the analyte is a nucleic acid, the at least one reagent comprises PCR primers and polymerase for performing PCR and the comprises a protocol for performing thermal cycling. In a further embodiment, the protocol is an enzyme assay, a binding assay, an immunoassay or PCR.

In yet another aspect of the invention, an instrument for performing a biological or chemical reaction is provided comprising: a unit comprising: an interface adapted to releasably engage a cartridge; the interface comprising means to receive a sample from an outlet of the cartridge and electronic reading means for reading a data storage component in the cartridge; and means for executing a protocol read from the data storage component. In one embodiment, the instrument further comprises a cartridge engaged with the interface, wherein the cartridge comprises: a first compartment adapted to receive a sample containing an analyte; a second compartment containing at least one reagent for performing a reaction on the analyte; an outlet; means for delivering the analyte and the at least one reagent from the outlet; and a data storage component comprising, in electronic form, a readable program for performing a reaction protocol on the analyte using the at least one reagent. In a further embodiment, the instrument of comprises a means for executing the protocol comprise a thermocycler adapted to perform PCR.

In another embodiment, a method is provided comprising: accepting a sample preparation device cartridge comprising: a compartment adapted to receive a sample; and an electronic data storage component; wherein the cartridge is configured to engage an interface of an instrument adapted to carry out a protocol; loading the compartment with a container containing an analyte; loading a protocol to perform a biological or chemical reaction using the analyte into the electronic storage component; and marketing the loaded cartridge to customers. In a further embodiment, the customers own said instrument. In a yet further embodiment, the reagents comprise PCR primers and polymerase for performing PCR and the protocol comprises a thermal cycling protocol. Furthermore, accepting comprises purchasing the cassette.

In another embodiment, a method is provided comprising: selling to an manufacturer a sample preparation device cartridge comprising: a compartment adapted to receive a reagent; and an electronic data storage component; wherein the cartridge is configured to engage an interface of an instrument adapted to carry out a protocol and wherein the cartridge is not loaded with the reagent or with electronic instructions to carry out a protocol involving the reagent; and selling the instrument to customers.

### EXAMPLE 1

### Obtaining Target Nucleic Acid Molecules

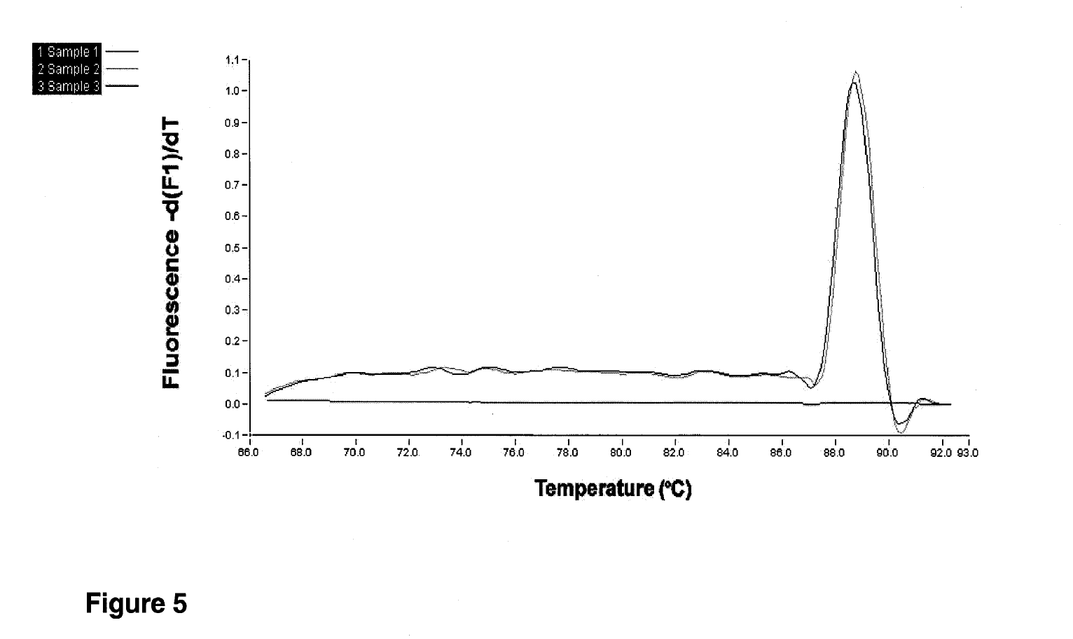

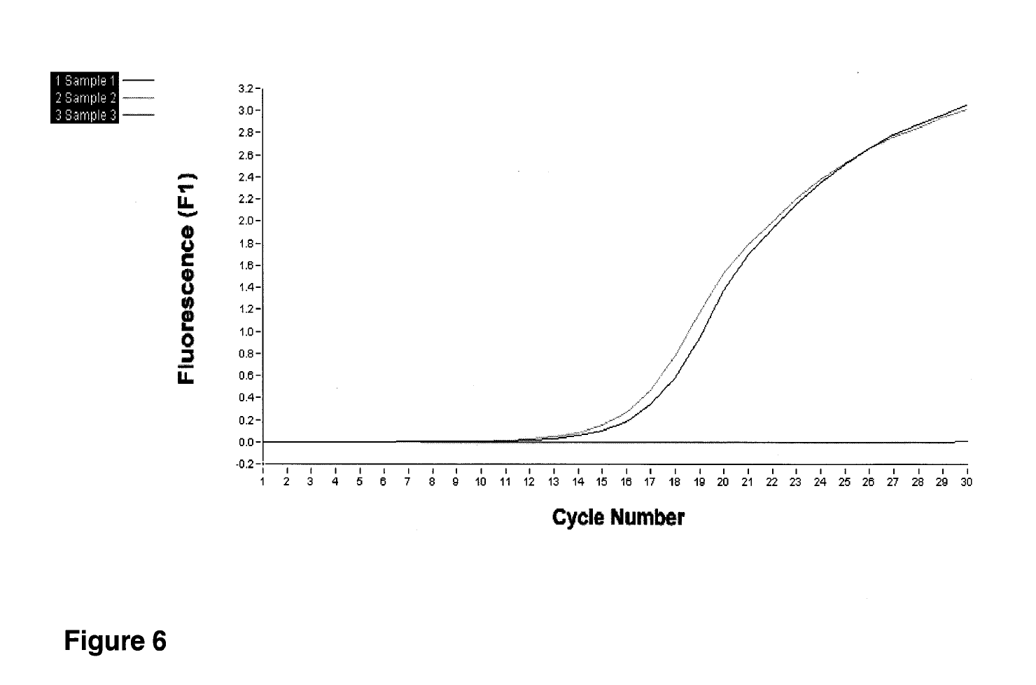

A sample (liquid supernatant from cell culture medium) was processed using a SPD and a conventional nucleic acid isolation column. The nucleic acid molecule isolated was non-replicating murine retro-viral vector carrying a GFP tag. More particularly, a liquid sample was split aliquot into two equal portions, one of which Sample 1 (FIGS. 5 and 6) was processed using a SPD (FIG. 1) and the second Sample 2 passed through a commercially available Qiagen spin column. As provided in FIGS. 5 and 6, the results demonstrate the efficiency of a SPD as compared to a conventional device. Therefore, the SPD provides a self-contained device, which provides efficient and rapid isolation of target nucleic acid molecules.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A sample preparation device comprising:
a housing comprising:
a) at least one first input port adapted to engage at least one or more pressure device adapted to deliver at least one fluid reagent into said first input port;
b) a processing component
c) at least one first channel in fluid communication between said at least one first input port and said processing component;
d) at least one second channel in fluid communication between said processing component and
i) a waste port, or
ii) a waste chamber;
e) at least one third channel in fluid communication between said processing component and at least one collection port; and
e) a valve having at least three positions, wherein the first position diverts fluid from the processing component to the waste port or waste chamber, the second position diverts fluid from the processing component to the collection port, and the third position prevents all flow from the processing component.

2. The sample preparation device of claim 1, wherein said housing is adapted to receive said processing component.

3. The sample preparation device of claim 1, wherein said processing component is integrated into said housing.

4. The sample preparation device of claim 1, wherein said valve is an only movable part in said housing.

5. The sample preparation device of claim 1, wherein said at least one or more pressure device is integrated into said housing.

6. The sample preparation device of claim 1, wherein said housing further comprises a second input port adapted to engage at least one second pressure device and a fourth channel in fluid communication between said second input port and said collection port without passing through said processing component.

7. The sample preparation device of claim 1, wherein said at least one or more pressure device is a syringe.

8. The sample preparation device of claim 7 wherein said housing one of said syringe comprises a dual chamber.

9. The sample preparation device of claim 1, wherein said at least one or more pressure device comprises a dual chamber.

10. The sample preparation device of claim 1 wherein said housing comprises a plurality of first input ports, each engaged with a pressure device.

11. The sample preparation device of claim 10 wherein each of said pressure devices comprise at least one different reagent.

12. The sample preparation device of claim 1 wherein said at least one collection port further comprises an outlet adapter that fits to a container.

13. The sample preparation device of claim 12, wherein said container is a collection vessel or reaction chamber.

14. The sample preparation device of claim 12, wherein said outlet adapter is releasable from said collection port.

15. The sample preparation device of claim 12 wherein said container is a capillary tube, conical tube, well, or PCR tube.

16. The sample preparation device of claim 1 wherein said processing component is adapted for nucleic acid purification, protein purification, or chemical compound purification.

17. The sample preparation device of claim 1 wherein said waste chamber is further linked to a waste port.

18. The sample preparation device of claim 1 wherein said waste chamber or waste port further comprises an aerosol filter.

19. The sample preparation device of claim 1, further comprising a data storage component.

20. The sample preparation device of claim 13, wherein said collection vessel or reaction chamber comprise reagents necessary for PCR.

21. The sample preparation device of claim 13, wherein said collection vessel or reaction chamber comprise lyophilized or gelified reagents.

22. The sample preparation device of claim 1, wherein one of said at least one or more pressure device comprises lyophilized or gelified reagents.

23. The sample preparation device of claim 1, wherein said device does not require cold storage.

24. The sample preparation device of claim 19, wherein said data storage capability comprises flash memory component, barcode or scannable label.

25. The sample preparation device of claim 6 comprising a plurality of pressure devices each engaged with respective input ports, wherein the plurality comprises: at least one positive pressure device adapted to receive a biological sample, at least one positive pressure device comprises a cell lysis buffer, at least one positive pressure devices comprises wash buffer and at least one positive pressure device comprises elution buffer; and optionally at least one positive pressure device comprises DNA primers and DNA polymerase.

26. A kit comprising the sample preparation device of claim 1;
integrated pressure devices for fluid delivery; and
wherein said device is sealed in a pouch.

27. A kit comprising the housing of claims 1;
syringes comprising reagents; and
wherein said housing is sealed pouch.

28. The kit of claims 26 or 27, wherein said kit does not require cold storage.

29. A method of isolating a nucleic acid comprising:
a) delivering a sample through a first input port of the module of claim 1 into the processing component;
b) lysing said sample and capturing one or more nucleic acids in said processing component;
c) washing said captured nucleic acids; and
d) extracting said nucleic acids from said processing component, and
e) collecting the extracted nucleic acids out the collection port.

30. The method of claim 29, further comprising the addition of a reaction mix to the extracted nucleic acids in said container.

31. A sample preparation device comprising:
a) a housing comprising:
i) a waste chamber
ii) a collection port;
b) at least 3 syringes adapted to deliver fluid into a processing component;
c) a processing component with a material to capture DNA in fluid communication with said syringes; and
d) a valve with at least three positions that can deliver fluid into a waste chamber or a collection port or prevent fluid delivery by sealing a fluidic circuit in said housing.

32. The sample preparation device of claim 31, further comprising an additional syringe in fluidic communication with the collection port.

33. The sample preparation device of claim 31, wherein said syringes are empty, or comprise a reagent selected from the group consisting of lysis buffer, wash buffer, elution buffer, and reaction reagents.

34. A sample preparation device comprising:
a housing comprising:
a) at least one first input port adapted to engage at least one reagent reservoir
b) a processing component
c) at least one first channel in fluid communication between said at least one first input port and said processing component;
d) at least one second channel in fluid communication between said processing component and
i) a waste port, or
ii) a waste chamber;
e) at least one third channel in fluid communication between said processing component and at least one collection port;
f) a valve having at least three positions, wherein the first position diverts fluid from the processing component to the waste port or waste chamber, the second position diverts fluid from the processing component to the collection port, and the third position prevents all flow from the processing component; and
g) at least one pressure port adapted to engage a pressure device adapted to deliver at least one fluid reagent into said first input port.

35. A sample preparation device comprising:
a housing comprising:
a) at least one first input port adapted to engage at least one pressure device adapted to deliver at least one fluid reagent into said first input port;
b) a processing component
c) at least one first channel in fluid communication between said at least one first input port and said processing component;
d) at least one second channel in fluid communication between said processing component and
i) a waste port, or
ii) a waste chamber;
e) at least one third channel in fluid communication between said processing component and at least one collection port; and
e) a valve having at least three positions, wherein the first position diverts fluid from the processing component to the waste port or waste chamber, the second position diverts fluid from the processing component to the collection port, and the third position prevents all flow from the processing component.
f) a component having data storage capacity (DSC).

36. The device of claim 35, wherein the DSC comprises computer executable logic configured for testing one or more reagents.

37. The device of claim 35, wherein the DSC comprises computer executable logic configured to operate a PCR device.

38. The device of claim 35, further comprising a collection vessel.

39. The device of claim 38, wherein said collection vessel is operably linked to a PCR device.

40. A method of rapid pathogen detection comprising:
processing a biological sample with the sample preparation device of claims 1 or 30 to obtain at least one nucleic acid molecule in a reaction solution;
delivering said reaction solution to a collection vessel;
and analyzing said at least one nucleic acid sequence in thermal cycler comprising an optical assembly.

41. The method of claim 40, wherein said collection vessel is operably linked to said sample preparation device and to a PCR machine.

42. The method of claim 40, wherein said detection is in less than about 35 minutes.

43. The method of claim 40, wherein said sample preparation device further comprises a data storage component (DSC) which stores data related to said analyzing.

44. The method of claim 40, wherein said sample preparation device further comprises a data storage component which is capable of providing instructions to operate a PCR machine wherein said sample preparation device further comprises a data storage component (DSC) which stores data related to said analyzing vice.

45. A method of distributing a sample preparation device (SPD) to a distributor; wherein said distributor provides one or more said SPD, wherein each of said SPD is configured to comprise all necessary reagents for isolation of a target compound; and wherein said SPD is configured for storage or transport by said distributor.

46. A method of distributing a sample preparation device (SPD) to a distributor; wherein said SPD comprises: all necessary reagents for processing a sample and obtaining a target compound; a data storage component (DSC) comprising computer executable logic designed to store and analyze data derived from said processing; wherein said computer executable logic alternatively further functions to provide instructions for operation of a PCR device configured to be operably coupled to said SPD.

47. A sample preparation device cartridge comprising:
a first compartment adapted to receive a sample containing an analyte;
a second compartment containing at least one reagent for performing a reaction on the analyte;
an outlet;
means for delivering the analyte and the at least one reagent from the outlet; and
a data storage component comprising, in electronic form, a readable program for performing a reaction protocol on the analyte using the at least one reagent.

48. The cartridge of claim 47 wherein the analyte is a nucleic acid, the at least one reagent comprises PCR primers and polymerase for performing PCR and the comprises a protocol for performing thermal cycling.

49. The cartridge of claim 48 wherein the protocol is an enzyme assay, a binding assay, an immunoassay or PCR.

50. An instrument for performing a biological or chemical reaction comprising:
a unit comprising:
an interface adapted to releasably engage a cartridge; the interface comprising means to receive a sample from an outlet of the cartridge and electronic reading means for reading a data storage component in the cartridge; and means for executing a protocol read from the data storage component.

51. The instrument of claim 50 further comprising a cartridge engaged with the interface, wherein the cartridge comprises:
a first compartment adapted to receive a sample containing an analyte;
a second compartment containing at least one reagent for performing a reaction on the analyte;
an outlet;
means for delivering the analyte and the at least one reagent from the outlet; and
a data storage component comprising, in electronic form, a readable program for performing a reaction protocol on the analyte using the at least one reagent.

52. The instrument of claim 50 wherein the means for executing the protocol comprise a thermocycler adapted to perform PCR.

53. A method comprising:
a) accepting a sample preparation device cartridge comprising:
i) a compartment adapted to receive a sample; and
ii) an electronic data storage component;
wherein the cartridge is configured to engage an interface of an instrument adapted to carry out a protocol;
b) loading the compartment with a container containing an analyte;
c) loading a protocol to perform a biological or chemical reaction using the analyte into the electronic storage component; and
d) marketing the loaded cartridge to customers.

54. The method of claim 53 wherein the customers own said instrument.

55. The method of claim 53 wherein the reagents comprise PCR primers and polymerase for performing PCR and the protocol comprises a thermal cycling protocol.

56. The method of claim 53 wherein accepting comprises purchasing the cassette.

57. A method comprising:
a) selling to a manufacturer a sample preparation device cartridge comprising:
i. a compartment adapted to receive a reagent; and
ii. an electronic data storage component;
wherein the cartridge is configured to engage an interface of an instrument adapted to carry out a protocol and wherein the cartridge is not loaded with the reagent or with electronic instructions to carry out a protocol involving the reagent; and
b) selling the instrument to customers.
